(19) United States
(12) Patent Application Publication
(10) Pub. No.: US 2022/0081732 A1
(43) Pub. Date: Mar. 17, 2022

(54) PROCESS FOR MAKING L-FUCOSE

(71) Applicant: DUPONT NUTRITION BIOSCIENCES APS, COPENHAGEN K (DK)

(72) Inventors: Jari LEWANDOWSKI, KANTVIK (FI); Juha NURMI, KANTVIK (FI); Hannu KOIVIKKO, KANTVIK (FI); Jyrki KUUSISTO, KANTVIK (FI); Nina NURMI, KANTVIK (FI); Jani SIITONEN, KANTVIK (FI); Juho JÄRVINEN, KANTVIK (FI)

(21) Appl. No.: 17/423,961

(22) PCT Filed: Jan. 20, 2020
(86) PCT No.: PCT/US20/14261

§ 371 Date: Jul. 19, 2021

(30) Foreign Application Priority Data
Jan. 21, 2019 (EP) 19152810.8

(51) Int. Cl. C13K 11/00; B01D 61/02; B01D 61/58; B01D 15/36; B01J 39/26

(52) U.S. Cl. CPC C13K 11/00; B01D 61/027; B01J 39/26; B01D 15/362; B01D 61/58

(57) ABSTRACT

This specification relates to a process for preparing fucose from a human milk oligosaccharide (“HMO”) comprising a fucose moiety, as well as L-fucose compositions prepared by such a process.

### CROSS-REFERENCE TO RELATED PATENT APPLICATION

This specification claims priority to European Patent Application 19152810.8 filed Jan. 21, 2019. The entire text of European Patent Application 19152810.8 is incorporated by reference into this specification.

### FIELD

This specification relates to the field of sugar separation technology, and, more particularly, to a process for preparing fucose from a human milk oligosaccharide (“HMO”) comprising a fucose moiety, as well as L-fucose compositions prepared by such a process.

### BACKGROUND

Fucose is found in a wide variety of natural products from many different sources, both in D- and L-form. Interest in L-fucose has recently increased because of its potential in the medical field in treating various disease conditions, such as tumours, inflammatory conditions and disorders relating to the human immune system. L-fucose also has applications in the cosmetic field, for instance as a skin moisturising agent.

In accordance with Merck Index, Twelfth Edition, 1996, crystalline L-fucose has a melting point of 140° C. and an optical rotation of −75.6°.

L-fucose has been recovered from natural sources and synthesized enzymatically, chemically or microbiologically. Known synthesis processes include multi-step processes with undesirably low L-fucose yields.

L-fucose is, for instance, a moiety in several human milk oligosaccharides.

Fucose also is associated with various plant polysaccharides, which are often highly branched structures having L-fucopyranosyl units either at the ends of or within the polysaccharide chains. In some cases, even methylated fucopyranosyl units occur in plant polysaccharides. L-fucose or methylated L-fucopyranosyl units occur in, for example, the cell walls of potato, cassava tuber and kiwi fruit, in the seed polysaccharides of soybean and in winged bean varieties and canola. Seaweed polysaccharides, found in the intercellular mucilage, form complex structures and are often composed of sulphated L-fucose polymers, named fucoidan.

Extracellular polysaccharides from various bacteria, fungi and micro-algae also contain L-fucose.

Efficient separation of L-fucose with a desirable high purity has been challenging.

U.S. Pat. No. 9,902,984, Glycom A/S, “Fermentative production of oligosaccharides” discusses a process of making a mixture of 2′-FL and DFL, which can be subjected to hydrolysis initiated by an acid or mediated by a fucosidase to produce fucose.

EP 1664352, DuPont Nutrition Biosciences ApS, “Separation of sugars” discusses a process of separating and recovering deoxy sugars from a biomass-derived solution. Several chromatographic separation steps are required to obtain an L-fucose fraction in sufficient purity to enable crystallization of L-fucose. It also discusses a crystallization process to produce high purity crystalline L-fucose from a solution containing galactose at less than 1% on DS.

EP 0102535, Hoechst Aktiengesellschaft, “Verfahren zur Herstellung von Rhamnose oder Fucose” discusses a process for making rhamnose or fucose from extracellular polysaccharides.

EP 2825545, Inalco S.R.L., “Process for the recovery of L-fucose from exopolysaccharides” discusses isolating L-Fucose from exopolysaccharides obtained by fermentative process.

WO2012/034996, Inalco S.p.A., “Process for production of L-fucose” discusses a process for making L-fucose by hydrolysis of a polysaccharide produced by a fermentation process effected by an isolated microbial strain.

WO 2018/180727, Yaizu Suisankagaku Industry, “Process for producing fucose-containing composition, and process for producing food and drink, cosmetic, toiletry goods, quasi-drug, and pharmaceutical containing fucose-containing composition” discusses a multiple step process for making fucose from polysaccharides. Crystalline fucose is reportedly obtained.

Saari et al. (Journal of Liquid Chromatography & Related Technologies, 32:14, 2050-2064, 2009) discusses a process for making L-fucose from hemicellulose hydrolysates.

The various known processes for producing and purifying L-fucose can, for example, be undesirably laborious. Consequently, there is an ongoing need for improved L-fucose manufacturing processes.

### SUMMARY

Briefly, this specification generally discloses a process for making fucose. It has been found that this process can be useful to address various disadvantages of prior known processes for making L-fucose.

In particular, this specification discloses, in part, a process for making L-fucose. The process comprises hydrolyzing a human milk oligosaccharide, which contains one or more L-fucose moieties in its structure. This hydrolysis forms a hydrolysate comprising fucose, lactose, galactose and glucose. The hydrolysate, in turn, is subjected to one or more purification steps comprising chromatographic separation and/or nanofiltration. A fucose-enriched fraction is recovered from the purification, which, in turn, is subjected to spray-drying and/or crystallization to form a purified fucose solid.

This specification also discloses, in part, a crystalline or spray-dried L-fucose product obtained from the above process.

The process generally comprises, for example, a combination of selective hydrolysis, fractionation by chromatography or nanofiltration (or a fractionation by a combination of chromatography and nanofiltration), and crystallization and/or spray-drying to make an L-fucose product from an HMO containing one or more L-fucose moieties in its structure.

The specification provides a versatile process for making fucose from HMOs, such as 2′-fucosyllactose, 3-fucosyllactose and/or difucosyllactose. In HMOs fucose generally exist in the L-form. The process of this specification is based on the selective hydrolysis of the HMO containing an L-fucose moiety, use of chromatographic fractionation and/or nanofiltration. After the chromatographic separation or nanofiltration, the fraction enriched in L-fucose may be further crystallized or spray-dried, and particularly crystallized to obtain L-fucose with high purity. Membrane filtration, like nanofiltration, can, for example, be used at any stage of the process to increase the purity where needed.

With the chromatographic process of this specification, for example an L-fucose fraction having a purity of from 60 to 98% (and typically from 80 to 95% or more) can generally be recovered in one chromatographic fractionation step. With the nanofiltration process of this specification, for example, an L-fucose permeate having a purity of from 50 to 90% (typically from 60 to 80% or more) generally can be obtained.

It has been found that crystallization following chromatographic separation and/or nanofiltration, as disclosed in this specification, can provide an L-fucose product having a purity of up to 99%, or greater, and a melting point of at least 140° C.

In some embodiments, the crystallization of L-fucose is carried out on a solution comprising a high galactose content of from 1 to 15%/DS, at high temperature. The crystallization process disclosed in this specification generally provides an industrially, economically and environmentally feasible crystallization process to produce high purity crystalline L-fucose regardless of excess of galactose.

In some embodiments, the crystallization solvent is water with no organic solvent being added or required. L-fucose crystallization from an aqueous solution can provide a solvent-free crystalline L-fucose product. This can be particularly useful for making fucose for medical applications.

It has been found L-fucose can generally be made from high-purity from HMOs, such as 2′-fucosyllactose (2′-FL), 3-fucosyllactose (3-FL) and difucosyllactose (LDFT). In some embodiments, side streams of an HMO downstream purification process can be used as a raw material to make the L-fucose. In some embodiments, the whole process for the recovery of L-fucose is carried out in an aqueous solution without the use of an organic solvent. The process, therefore, may be carried out with fewer process steps than various known processes for recovering L-fucose. Absence of an organic solvent also means, for example, less chemicals are generally used to make the high purity L-fucose.

The process of this specification also provides a crystalline L-fucose with high purity. The crystallization of L-fucose may be carried out from the L-fucose-containing fraction obtained from the chromatography or membrane filtration. In some embodiments, the crystallization of L-fucose comprises a crystallization from water (and without any organic solvent) resulting in crystalline L-fucose with a high purity and with a high yield. Thus, this process generally can be beneficial as a sustainable and economic L-fucose production process.

Further benefits of the teachings of this specification will be apparent to one skilled in the art from reading this specification.

### BRIEF DESCRIPTION OF THE DRAWINGS

FIG. 1 is a graphical presentation of the elution profile according to Example 19 (chromatographic fractionation of 2′-FL crystallization mother liquor hydrolysate with a strong acid cation exchange resin in Na⁺ form).

FIG. 2 is a microscopic image of the crystal mass of Example 25 after 38 hr from seeding from crystallization at high temperature of from 75 to 44° C., 40-fold magnification representing large crystal size and cubic crystal habit. Typically, the ratio of the largest and the second largest dimension is between 1 and 2.

FIG. 3 is a microscopic image of the crystal mass of Example 26 after 41 hr from seeding from crystallization at low temperature of from 55 to 25° C., 40-fold magnification representing small crystal size and needlelike crystal habit. Typically, the ratio of the largest and the second largest dimension is between 2 and 5.

### DETAILED DESCRIPTION

This detailed description is intended only to acquaint others skilled in the art with Applicant's invention, its principles, and its practical application so that others skilled in the art may adapt and apply the invention in its numerous forms, as they may be best suited to the requirements of a particular use. This detailed description and its specific examples, while indicating certain embodiments, are intended for purposes of illustration only. This specification, therefore, is not limited to the described embodiments, and may be variously modified.

The process of this specification may comprise, for example, a combination of (i) selective hydrolysis, (ii) fractionation by chromatography and/or membrane filtration, and (iii) crystallization and/or spray-drying to make L-fucose from an HMO containing one or more L-fucose moieties in its structure. Using a process of this specification, it has been found L-fucose can be manufactured at industrial scale in high yield and high purity from starting material containing 2′-fucosyllactose (2′-FL), 3-fucosyllactose (3-FL) and/or difucosyllactose (LDFT) without laborious and costly purification steps of other processes. In some embodiments, the whole process for making L-fucose is carried out in an aqueous solution without the use of an organic solvent.

The starting material may be selected from, for example, liquor originating from fermentation containing 2′-FL, 3-FL and/or LDFT; 2′-FL, 3-FL and/or LDFT crystalline or spray dried product; purified 2′-FL, 3-FL and/or LDFT syrup; 2′-FL, 3-FL and/or LDFT crystallization mother liquor; and/or other solutions containing fuicosylated lactoses.

### Feed Materials

A starting material in a process of this specification comprises an HMO, which comprises one or more L-fucose moieties in its structure. The starting material may be, for example, a solution derived from a fermentation containing 2′-FL, 3-FL and/or LDFT; 2′-FL, 3-FL and/or LDFT crystalline or spray-dried product; purified 2′-FL, 3-FL and/or LDFT syrup; and/or 2′-FL, 3′-FL and/or LDFT crystallization mother liquor. The starting material may also be, for example, an intermediate product (in the form of, for example, a syrup or spray-dried product) or side stream from an HMO manufacturing process, such as a side stream from a 2′-FL, 3-FL and/or LDFT manufacturing process. In addition to the HMO comprising one or more L-fucose moieties in its structure, the starting material may comprise, for example, lactose, L-fucose and one or more other oligosaccharides (each of which may also be, for example, an HMO comprising one or more L-fucose moieties in its structure).

### Purities and Yields

In some embodiments, the L-fucose yield from the HMO hydrolysis is more than 50% relative to the theoretical L-fucose yield based on the starting material. In some embodiments, the L-fucose yield from the hydrolysis is more than 60% relative to the theoretical L-fucose yield based on the starting material. In some embodiments, the L-fucose yield from the hydrolysis is more than 70% relative to the theoretical L-fucose yield based on the starting material. In some embodiments, the L-fucose yield from the hydrolysis is more than 75% relative to the theoretical L-fucose yield based on the starting material. In some embodiments, the L-fucose yield from the hydrolysis is more than 80% relative to the theoretical l-fucose yield based on the starting material.

In addition to the forming L-fucose, the HMO hydrolysis generally forms lactose, which, in turn, can further hydrolyze to glucose and galactose. In general, the hydrolysis is conducted under conditions that selectively minimize the amount of lactose hydrolyzed to glucose and galactose. In some embodiments, the galactose and glucose yields from the hydrolysis are less than 20% based on the lactose that could potentially be formed during the hydrolysis (and, in turn, be hydrolyzed into glucose and galactose). In some embodiments, the galactose and glucose yields from the hydrolysis are less than 15% based on the lactose that could potentially be formed during the hydrolysis. In some embodiments, the galactose and glucose yields from the hydrolysis are less than 12% based on the lactose that could potentially be formed during the hydrolysis. In some embodiments, the galactose and glucose yields from the process are less than 10% based on the lactose that could potentially be formed during the hydrolysis.

In some embodiments wherein chromatographic separation is used, the L-fucose fraction recovered from the chromatographic separation has a purity of more than 65% on DS. In some embodiments, the L-fucose fraction recovered from the chromatographic separation has a purity of more than 70% on DS. In some embodiments, the L-fucose fraction recovered from the chromatographic separation has a purity of more than 80% on DS. In some embodiments, the L-fucose fraction recovered from the chromatographic separation has a purity of more than 90% on DS. In some embodiments, the process provides an L-fucose yield of more than 70%. In some embodiments, the chromatographic separation provides an L-fucose yield of more than 80%. In some embodiments, the chromatographic separation provides an L-fucose yield of at least 90%.

In some embodiments wherein nanofiltration is used, the yield of L-fucose from the nanofiltration is more than 70% based on L-fucose present in the hydrolysate. In some embodiments, the yield of L-fucose in the nanofiltration is more than 80% based on the fucose present in the hydrolysate. In some embodiments, the yield of L-fucose in the nanofiltration is more than 90% based on the fucose present in the hydrolysate. In some embodiments, L-fucose content in the purified sugar syrup (permeate) is more than 50% on DS. In some embodiments, L-fucose content in the purified sugar syrup (permeate) is more than 60% on DS. In some embodiments, L-fucose content in the purified sugar syrup (permeate) is more than 70% on DS. In some embodiments, L-fucose content in the purified sugar syrup (permeate) is more than 80% on DS.

In some embodiments, the crystallization provides crystalline L-fucose having a purity of more than 90%. In some embodiments, the crystallization provides crystalline L-fucose having a purity of more than 95%. In some embodiments, the crystallization provides crystalline L-fucose having a purity of more than 99%. In some embodiments, the crystallization process provides an L-fucose yield of more than 30%. In some embodiments, the crystallization process provides an L-fucose yield of more than 40%. In some embodiments, the crystallization process provides an L-fucose yield of more than 50%. In some embodiments, the crystallization process provides an L-fucose yield of more than 60%.

### Hydrolysis

During the hydrolysis, the starting material is hydrolyzed to release L-fucose in monomeric form in high yield without substantially hydrolyzing lactose. In some embodiments, the hydrolysis step is carried out as a selective hydrolysis by adjusting the hydrolysis conditions (temperature, pH and hydrolysis time) so that an optimal release of L-fucose in relation to galactose and glucose and other sugars is achieved using an acid, enzyme or strong acid cation ion exchange resin in He-ion form. Selective hydrolysis provides a mixture where 2′-FL, 3-FL and/or LDFT is hydrolyzed mainly to L-fucose and lactose, and the lactose is not substantially further hydrolyzed. The minimization of lactose hydrolysis generally facilitates, for example, the subsequent chromatographic separation and nanofiltration steps.

In some embodiments, with selective hydrolysis, the galactose and glucose yields are less than 20% (based on the potential lactose that could be formed from the starting material, and, thus, further hydrolyzed to glucose and galactose). In some embodiments, with selective hydrolysis, the galactose and glucose yields are less than 15% (based on the potential lactose that could be formed from the starting material, and, thus, further hydrolyzed to glucose and galactose). In some embodiments, with selective hydrolysis, the galactose and glucose yields are less than 12% (based on the potential lactose that could be formed from the starting material, and, thus, further hydrolyzed to glucose and galactose). In some embodiments, with selective hydrolysis, the galactose and glucose yields are less than 10% (based on the potential lactose that could be formed from the starting material, and, thus, further hydrolyzed to glucose and galactose). Potential lactose refers to lactose that would be formed if the hydrolysis of the HMO in the starting material would be complete. It should be understood that, for selective hydrolysis, conditions are generally controlled in such a way that the hydrolysis of the HMO in the starting material is not complete.

In some embodiments, L-fucose yield from the selective hydrolysis is more than 75% of the theoretical yield based on the starting material. And, in some such embodiments, the galactose and glucose yields are less than 12% (based on the potential lactose that could be formed from the starting material and, thus, further hydrolyzed to glucose and galactose). In some embodiments, L-fucose yield from the selective hydrolysis is more than 80% of the theoretical yield based on the starting material. And, in some such embodiments, the galactose and glucose yields are less than 10% (based on the potential lactose that could be formed from the starting material, and, thus, further hydrolyzed to glucose and galactose).

In some embodiments, the selective hydrolysis is conducted at a pH that favors a high L-fucose yield, while also minimizing the amount of galactose and glucose formed. In some embodiments, the hydrolysis is performed (at least partly, or, alternatively, entirely) at a pH of from 1.0 to 3.0; or, alternatively, at a pH of from 1.0 to 2.0; or, alternatively, at a pH of from 1.5 to 2.0; or, alternatively, at a pH of from 1.5 to 1.75. pH is generally selected to suit with the temperature and duration of the acid hydrolysis.

In some embodiments, a pH of about 2.0 is used during the hydrolysis, and the resulting L-fucose yield from L-fucose moieties in the starting solution is more than 50% of the theoretical yield and the resulting glucose and galactose yields from the lactose is less than 5% of the theoretical yield. In some embodiments, a pH of about 1.75 is used during the hydrolysis, and resulting the L-fucose yield from L-fucose moieties present in the starting solution is more than 70% of theoretical yield and the resulting glucose and galactose yields are less than 5% out of theoretical yield. In some embodiments, a pH of about 1.5 is used during the hydrolysis, and the resulting L-fucose yield from L-fucose moieties in the starting solution is more than 80% of theoretical yield and the resulting glucose and galactose yields from the lactose are less than 15% of the theoretical yield. In some embodiments, a pH of about 1.0 is used during the hydrolysis, and the resulting L-fucose yield from L-fucose moieties present in the starting solution is more than 80% out of theoretical yield and the resulting glucose and galactose yields from lactose are less than 40% of the theoretical yield.

Selective hydrolysis with an enzyme may be effected with a suitable enzyme that hydrolyzes a target linkage. Fucosidase can release L-fucose from 2′-FL, 3-FL and/or LDFT very selectively to a mixture of L-fucose and lactose. Enzymes having 1,2-α-L-fucosidase activity and 1,3/4-α-L-fucosidase activity are examples of generally suitable enzymes to produce monomeric L-fucose. In some embodiments, the enzyme dose used is from 5 to 10 IU/g of HMO. The enzymatic hydrolysis is performed at a temperature selected based on enzyme stability, typically at temperatures of from 40 to 70° C. In some embodiments, the enzymatic hydrolysis is continued for from 3 to 48 hr; or, alternatively, from 3 to 16 hr. In some embodiments, the enzymatic hydrolysis is performed at a temperature of from 35 to 45° C. for 24 hr. The temperature and duration of the enzymatic hydrolysis is selected according to, for example, the enzyme dose used and enzyme stability.

Hydrolysis of 2′-FL, 3-FL and/or LDFT may also be performed by treating the solution with strong acid cation (SAC) ion exchange resin in H⁺-ion form. Ion exchange resin treatment can be done in a column. In some embodiments, the hydrolysis using ion exchange resin is carried out at a dry solids content of about 30%/DS. In some embodiments, the hydrolysis using ion exchange resin is carried out at a dry solids content of about 40%/DS. In some embodiments, the hydrolysis using ion exchange resin is carried out at a dry solids content of about 45%/DS. In some embodiments, the hydrolysis using SAC resin in H⁺-ion form is performed at a feed pH of from 2.5 to 7.0. In some embodiments, the hydrolysis using SAC H⁺ resin is performed at a temperature of 60° C.; or, alternatively, 70° C.; or, alternatively, 75° C.; or, alternatively, 80° C. In some embodiments, the flow rate in the hydrolysis using SAC H+ resin is from 0.1 to 10 BV/h; or, alternatively, from 0.1 to 5 BV/h; or, alternatively, from 0.2 to 2.0 BV/h. In some embodiments, the flow rate is 0.1 BV/h; or, alternatively, 0.2 BV/h; or, alternatively, 0.25 BV/h; or, alternatively, 0.5 BV/h; or, alternatively, 1.0 BV/h; or, alternatively, 2.0 BV/h. Hydrolysis degree using SAC (H+) column depends on, for example, the feed solution contact time with the SAC (H+) resin, temperature, feed solution cation type and concentration, and feed solution pH.

In some embodiments, the hydrolysis using SAC H⁺ ion exchange resin is performed at a temperature of from 60 to 100° C. (or, alternatively, from 70 to 80° C.); a pH of from 2.5 to 7.0; and a flow rate of from 0.1 to 2.0 BV/h (or, alternatively, from 0.2 to 1.0 BV/h).

The hydrolysis is typically carried out as acid hydrolysis with an inorganic acid, such as, for example, sulphuric acid, sulphurous acid or hydrochloric acid; or with an organic acid, such as, for example, acetic acid, formic acid or oxalic acid.

In some embodiments, the acid hydrolysis temperature is from 70 to 140° C. (or, alternatively, from 80 to 110° C.), and the hydrolysis time is from 0.5 to 6 hr. In some embodiments, the acid hydrolysis is typically carried out at a pH of from 0.5 to 2.5; or, alternatively, from 1.0 to 2.0; or, alternatively, from 1.5 to 1.75. In some embodiments, the acid hydrolysis is carried out at a temperature of from 90 to 100° C. and pH of from 1.1 to 2.0, and the hydrolysis is continued for from 1 to 3 hr. In some embodiments, the amount of acid used for the hydrolysis is from 0.2 to 0.6%/DS of 100% acid.

The amount of the acid used in the selective hydrolysis generally depends on the hydrolysis temperature: a lower temperature tends to require a greater amount of acid and/or a longer reaction time, and a greater temperature tends to require a lower amount of acid and/or a shorter reaction time. When pure solutions are used, there is practically no buffer in the solution and only a small amount of acid is generally needed. In some embodiments, acid hydrolysis is carried out at a pH of from 1.0 to 2.0 at temperature of from 85 to 96° C. for from 2 to 4 hr. In some embodiments, acid hydrolysis is carried out at a pH of from 1.5 to 1.75 and temperature of from 85 to 95° C. for from 2 to 4 hr. In some embodiments, acid hydrolysis is carried out at a pH of from 1.4 to 1.5 and a temperature of from 88 to 96° C. for 100 min. In some embodiments, the acid hydrolysis is carried out at a pH 1.5 to 1.75 and a temperature of from 85 to 96° C. for from about 2 to 4 hr.

In some embodiments, the dry substance content of the hydrolysate is from 10 to 70% by weight; or, alternatively, from 20 to 65% by weight; or, alternatively, from 40 to 60% by weight.

In some embodiments, the hydrolysis conditions are typically selected so that more than 50% (or, alternatively, more than 60%; or, alternatively, more than 70%; or, alternatively, more than 80%) out of theoretical L-fucose yield of the 2′-FL, 3-FL and/or LDFT present in the starting material is hydrolysed into monomeric L-fucose.

In some embodiments, the hydrolysis conditions are selected so as to obtain a hydrolysate in which the content of L-fucose is at least 10% on DS; or, alternatively, more than 15% on DS; or, alternatively, more than 20% on DS; or, alternatively, more than 25% on DS.

In some embodiments, the hydrolysis conditions are selected to obtain a hydrolysate where the content of galactose and glucose is less than 20% on DS; or, alternatively, less than 15%; or, alternatively, less than 10% on DS; or, alternatively, less than 5% on DS.

The selective hydrolysis may be carried out as a batch process or as a continuous process. The hydrolysis vessel may be, for example, a mixed reactor or a tubular reactor, optionally provided with a continuous flow.

When the selective hydrolysis is carried out as an acid hydrolysis, the hydrolysis step is typically followed by neutralization. Neutralization may be carried out with any useful alkali, such as, for example, CaO, MgO, NaOH, KOH, Na₂CO₃ or CaCO₃. In some embodiments, neutralization is carried out with NaOH or KOH. In some embodiments, the pH is adjusted to at least about pH 2.0 at neutralization.

The reagents used for the hydrolysis and neutralization typically introduce various salts into the hydrolysate. The salts may be essentially (or completely) removed from the hydrolysate in subsequent fractionation steps of the process.

After the hydrolysis, the undissolved solids may be separated from the aqueous hydrolysate in a known manner, such as filtration, to obtain a clarified hydrolysate.

Hydrolysate from an acid hydrolysis of this specification may comprise, for example, L-fucose at from 15 to 35%; lactose at from 15 to 65%; galactose and glucose at from 1 to 10%; and, depending on the starting material, 2′-FL and/or 3-FL at from 0 to 10% and LDFT at from 0 to 5%. When the hydrolysis of LDFT is conducted with an enzyme, the hydrolysate may comprise, for example, LDFT at from 0 to 10% in addition to the components mentioned above, with essentially no galactose and glucose present. When hydrolysis of LDFT is conducted with H+-ion form strong acid cation ion exchange resin, the hydrolysate may comprise, for example, 0 to 10% LDFT in addition to the components mentioned above.

### Chromatographic Separation

The hydrolysis product generally comprises L-fucose and at least one other monosaccharide (e.g., galactose and/or glucose); poly-, oligo- and/or disaccharides (e.g., lactose, 2′-FL, 3-FL and LDFT); and salts from the acid hydrolysis and neutralization. In some embodiments, this product is subjected to chromatographic separation (also referred to as chromatographic fractionation). A fractionation generally provides a fraction enriched in L-fucose, and at least one other fraction selected from the group consisting of (i) a fraction enriched in lactose and other low molecular weight components of the feed solution (e.g., galactose and/or glucose); and (ii) one or more fractions enriched in poly-, oligo- and/or disaccharides and soluble polymers. The fractionation is followed by the recovery of the fraction enriched in L-fucose, and, optionally, one or more of the other fractions.

Surprisingly, one chromatographic separation is generally sufficient to recover L-fucose fraction in high purity and yield. The fraction enriched in L-fucose typically contains at least 50% L-fucose on DS, less than 25% on DS of one or more monosaccharides selected from galactose and glucose, and less than 20% lactose on DS. In some embodiments, the fraction enriched in L-fucose contains at least 75% L-fucose on DS, less than 15% on DS of one or more monosaccharides selected from galactose and glucose, and less than 10% lactose on DS. In some embodiments, the fraction enriched in L-fucose contains at least 90% L-fucose on DS, less than 10% on DS of one or more monosaccharides selected from galactose and glucose, and less than 1% lactose on DS.

In some embodiments, the L-fucose content in the purified sugar syrup (L-fucose enriched fraction) is more than 60% on DS; or, alternatively, more than 70% on DS; or, alternatively, more than 80% on DS; or, alternatively, more than 90% on DS. In some embodiments, the L-fucose yield in the chromatographic separation is more than 70% on DS; or, alternatively, more than 80% on DS; or, alternatively, from more than 90% on DS.

In some embodiments, the chromatographic fractionation is carried out using a column packing material selected from cation exchange resins and anion exchange resins. The resins may, for example, be in a macroporous form or a gel form. In some embodiments, the resin is in a gel form.

In some embodiments, the chromatographic fractionation is carried out with cation exchange resins. The cation exchange resins may be selected from strong acid cation exchange resins and weak acid cation exchange resins.

### Strong Acid Cation Exchange Resins (SAC Resins)

The SAC resins generally may have, for example, a styrene or acrylic skeleton. In some embodiments, the resin is a sulphonated polystyrene-co-divinylbenzene resin. Other alkenyl aromatic polymer resins, like those based on monomers like alkyl-substituted styrene or mixtures thereof, can also typically be applied. The resin may also be crosslinked with other suitable aromatic crosslinking monomers, such as, for example, divinyltoluene, divinylxylene, divinylnaphtalene or divinylbenzene (DVB); or with aliphatic crosslinking monomers, such as isoprene, ethylene glycol diacrylate, ethylene glycol dimethacrylate, N,N′-methylene bis-acrylamide or mixtures thereof. The cross-linking degree of the resin is typically from about 1 to about 20% (or, alternatively, from about 3 to about 8%) of the cross-linking agent, such as divinyl benzene.

The SAC resins used for the chromatographic separation may be in a multivalent, divalent or monovalent cation form.

The monovalent cation forms may be selected from, for example, H⁺, Na⁺ and K⁺. Examples of divalent cation forms are Ca²⁺, Mg²⁺, Zn²⁺, Sr²⁺ and Ba²⁺. And an example of a trivalent cation form is Al³⁺.

In some embodiments, the SAC resin used for the chromatographic separation is in a monovalent or divalent cation form. In some embodiments, the SAC resin is in a Na⁺ or Ca²⁺ form. In some embodiments, the SAC resin is in a H⁺-ion form.

A typical mean average particle size of the resin is from 10 to 2000 μm. In some embodiments, the mean average particle size of the resin is from 100 to 400 μm.

### Weak Acid Cation Exchange Resins (WA Resins)

A WAC resin is generally an acrylic cation exchange resin having carboxylic functional groups.

The acrylic WAC resin is typically derived from the group consisting of an acrylate ester, acrylonitrile, acrylic acids and mixtures thereof. Typically suitable acrylate esters include those selected from the group consisting of methyl methacrylate, methyl acrylate, ethyl acrylate and butyl acrylate.

The matrix of the WAC resins may also be other than acrylic.

The active functional groups of the WAC resins may also be other than carboxylic groups. They may be selected from, for example, other weak acids.

A WAC resin may be in a H⁺, Na⁺, K⁺, Ca²⁺ or Mg²⁺ form. In some embodiments, the WAC resin is in a H⁺ or Na⁺ form. In other embodiments, other ion forms are used.

A WAC resin is generally crosslinked with an aromatic crosslinker. In some embodiments, the crosslinker comprises divinylbenzene (DVB). It may also be crosslinked with an aliphatic crosslinker, such as, for example, isoprene, 1,7-octadiene, trivinylcyclohexane, diethylene glycol divinylether. In some embodiments, the crosslinking degree is from 1 to 20% DVB; or, alternatively, from 3 to about 8% DVB.

In some embodiments, the average particle size of the WAC resin is from 10 to 2000 μm; or, alternatively, from 100 to 400 μm.

### Separation Conditions

The eluent for the chromatographic separation may, for example, be selected from water, an aqueous solution, an alcohol, an evaporation condensate and mixtures thereof. In some embodiments, the eluent is water.

In some embodiments, the separation is performed at a temperature of from 20 to 95° C.; or, alternatively, from 60 to 80° C.

In some embodiments, the solution used as the feed has an acidic pH, such as from 2 to 7.

In some embodiments, the separation is performed by a process selected from a simulated moving bed process and a batch process.

The simulated moving bed process may generally be performed by a sequential process or a continuous process or a combination thereof.

In some embodiments, other purification steps are performed in addition to the chromatographic separation. Such steps may occur before and/or after the chromatographic separation, In some embodiments, the recovered L-fucose fraction from the chromatographic separation is subjected to one or more further steps, such as, for example, evaporation, concentration, filtration, ion exchange, active carbon treatment, sterile filtration, crystallization, intermediate crystallization and nanofiltration. The recovered L-fucose fraction(s) from the chromatographic separation may be treated in different ways, depending on the purity of the fraction(s) and the desired purity of the final product.

### Nanofiltration

Fractionation of the hydrolysate may additionally or alternatively be carried out by membrane filtration, selected from, for example, ultrafiltration and nanofiltration. In some embodiments, the membrane filtration comprises nanofiltration. Nanofiltration is a pressure-driven membrane filtration-based process. The nanofiltration provides two fractions: (i) a retentate enriched in di-, poly- and/or oligosaccharides; and (ii) a permeate enriched in L-fucose.

In some embodiments, a hydrolysate produced by enzymatic hydrolysis is used as a feed for nanofiltration to obtain a permeate with a high L-fucose content and only small amount of other monomeric sugars (e.g., galactose and/or glucose).

In some embodiments, the nanofiltration permeate is collected in one or in several fractions, while the nanofiltration retentate is collected in one fraction.

In some embodiments, the nanofiltration is carried out as a batch process or a continuous process.

The nanofiltration is typically carried out at a temperature of from 5 to 80° C.; or, alternatively, from 30 to 75° C.; or, alternatively, from 50 to 70° C. In some embodiments, the nanofiltration is carried out at a pressure of from 5 to 60 bar; or, alternatively, from 10 to 50 bar; or, alternatively, from 20 to 45 bar. In some embodiments, the pH of the solution being subjected to nanofiltration is from 1 to 10; or, alternatively, from 2 to 8; or, alternatively, from 3 to 6. The desired pH generally depends on, for example, the composition of the starting solution, the membrane used for the nanofiltration, and the stability of the components to be recovered. If necessary, the pH of the starting solution may be adjusted to a desired value before nanofiltration.

In some embodiments, the nanofiltration is carried out with a flux of from 1 to 100 l/m²h (or, alternatively, with a flux of from 2 to 50 l/m²h; or, alternatively, with a flux of from 3 to 12 l/m²h). The flux at which the nanofiltration is carried out generally depends on, for example, the concentration and viscosity of the nanofiltration feed.

In some embodiments, the nanofiltration membrane is selected from polymeric and inorganic membranes having MgSO₄ retention of from 50 to 99% (at 25° C., 2 g/l concentration, 8 bar, and a pH of 6); or, alternatively, from 70 to 99% (at 25° C., 2 g/l concentration, 8 bar, and a pH of 6); or, alternatively, from 96 to 99% (at 25° C., 2 g/l concentration, 8 bar, and a pH of 6); or, alternatively, from 98 to 99% (at 25° C., 2 g/l concentration, 8 bar, and a pH of 6).

In some embodiments, the membrane has a molecular weight cut-off (MWCO) of from about 100 to about 500 Daltons. In some embodiments, the membrane has an MWCO of from about 150 to about 400 Daltons. In some embodiments, the membrane has an MWCO of from about 150 to about 300 Daltons.

In some embodiments, the membrane has an MWCO of from about 100 to about 900 Daltons and a MgSO₄ retention of from about 50 to 99% at 25° C. In some embodiments, the membrane has an MWCO of from about 150 to about 500 Daltons and a MgSO₄ retention of about 80-99% at 25° C. In some embodiments, the membrane has an MWCO of from about 150 to about 300 Daltons and a MgSO₄ retention of from about 98 to 99% at 25° C.

The nanofiltration membrane may have a negative or positive charge. In some embodiments, the membrane is an ionic membrane (i.e., it may contain cationic or anionic groups). In some embodiments, the membrane is a neutral membrane. The nanofiltration membrane may be selected from hydrophobic and hydrophilic membranes.

In some embodiments, the nanofiltration membrane comprises a spiral wound membrane. Alternatively, the membrane configuration may be, for example, a flat sheet, tube or hollow fiber. “High shear” membranes, such as vibrating membranes and rotating membranes also can generally be used. The membrane can be tubular, spiral or flat in shape.

Before the nanofiltration begins, the nanofiltration membrane may be pretreated, such as with, for example, an alkaline detergent or ethanol. The membrane also may be washed with, for example, an alkaline detergent or ethanol during the nanofiltration process, if necessary.

In some embodiments, the nanofiltration equipment comprises at least one nanofiltration membrane element dividing the feed into a retentate and permeate section. Nanofiltration equipment typically also includes a means for controlling the pressure and flow, such as pumps and valves and flow and pressure meters and controllers. The equipment may also include several nanofiltration membrane elements in one pressure vessel in different combinations, arranged in parallel or in series.

In some embodiments, the yield of L-fucose from the nanofiltration is more than 70% (or, alternatively, more than 80%; or, alternatively, more than 90%) based on the L-fucose present in the hydrolysate. In some embodiments, the L-fucose content in the purified sugar syrup (permeate) from the nanofiltration is more than 50% on DS; or, alternatively, more than 60% on DS; or, alternatively, more than 70% on DS; and or, alternatively, more than 80% on DS.

The nanofiltration permeate may be subjected to further enzymatic hydrolysis to hydrolyse lactose to galactose and glucose. This may, for example, be helpful to facilitate a subsequent crystallization step.

A fractionation by membrane filtration may be used with one or more other purification steps, such as ion exchange, chromatographic separation, evaporation and filtration. These further purification steps may be carried out before or after the membrane filtration.

Furthermore, the recovered L-fucose fractions may be subjected to one or more further steps, such as evaporation, concentration, filtration, ion exchange, active carbon treatment, sterile filtration, crystallization, intermediate crystallization, nanofiltration and chromatographic fractionation. The recovered L-fucose fraction(s) may be treated in different ways, depending on the purity of the fractions.

In some embodiments, the permeate collected from the nanofiltration is subjected to subsequent enzymatic hydrolysis. In some embodiments, the permeate collected from the nanofiltration is subjected to hydrolysis by a lactase enzyme to hydrolyse lactose in the permeate to facilitate subsequent crystallization.

### Crystallization

In some embodiments, the L-fucose fraction obtained from chromatographic fractionation and/or membrane filtration is subjected to crystallization to obtain crystalline L-fucose.

The crystallization of L-fucose may be carried out by a traditional process, such as cooling crystallization or precipitation crystallization at a temperature of from 10 to 80° C. The crystallization of L-fucose may also advantageously be carried out by a boiling crystallization process or a boiling-and-cooling crystallization process.

In some embodiments, the fucose crystallization is carried out from a solution having an L-fucose purity of more than 60% on DS; or, alternatively, more than 70% on DS; or, alternatively, more than 80% on DS; or, alternatively, more than 90% on DS; or, alternatively, more than 95% on DS. In some such embodiments, the crystallization provides a crystalline L-fucose product having a purity of more than 90% on DS; or, alternatively, more than 95% on DS; or, alternatively, more than 99% on DS.

A combination of two or more of the crystallizations may be used.

In some embodiments, the crystallization is carried out using a solvent selected from water, alcohol (e.g., ethanol), or a mixture thereof. In some embodiments, the crystallization is carried out from water.

In some embodiments, the L-fucose crystallization is carried out by cooling crystallization. In some such embodiments, the solution containing L-fucose is first evaporated to an appropriate dry substance content (e.g., to an RDS of from about 60 to 90%) depending on the L-fucose content of the solution. The supersaturated solution may be seeded with seed crystals of L-fucose. The seeds, if used, are generally pulverized crystals in a dry form, or they are suspended in a crystallization solvent, which may be water, an alcohol (e.g., ethanol), or a mixture thereof. In some embodiments crystallization solvent is water. The crystallization mass is subjected to cooling (after seeding, if seeding is used) with simultaneous mixing until the crystallization yield and viscosity is optimal for the separation of crystals. In some embodiments, the cooling time is from 10 to 60 hr. In some embodiments, the temperature drop during cooling is from 5 to 40° C. Some additional crystallization solvent may be added during cooling to improve the crystallization yield or the crystal separation performance. The crystallization mass may then be mixed at the final temperature for a period of time (in some embodiments, from 0.5 to 24 hr) to reach the maximum crystallization yield. The crystals may then separated from the mother liquor by, for example, by filtration or centrifugation. The crystal cake may be washed with a liquid (in some embodiments, the liquid being the crystallization solvent), and, optionally, dried to obtain a high-purity product.

In some embodiments, the L-fucose crystallization is carried out by boiling crystallization combined with cooling crystallization. In some such embodiments, the solution containing L-fucose is first evaporated to supersaturation at the boiling point of the solution. The solution is then seeded (if seeding is used), and the evaporation is continued at the boiling point of the crystallization mass (i.e., the mixture of the supersaturated solution and crystals) to obtain improved crystal size distribution and yield, until a crystallization mass is obtained in which the crystal yield is from 1 to 60% on L-fucose, and the dry solids content of the mass is greater than 60% by weight. In some embodiments, the evaporation is carried out at a temperature of from 50 to 70° C. After boiling crystallization, the crystallization mass is subjected to cooling with simultaneous mixing until the crystallization yield and viscosity is optimal for the separation of crystals. In some embodiments, the cooling time is from 10 to 60 hr. In some embodiments, the temperature drop during cooling is from 5 to 40° C., depending on the boiling crystallization yield and the crystal size distribution. Additional crystallization solvent may be added during cooling to further improve the crystallization yield and the crystal separation performance. The crystallization mass may then be mixed at the final temperature for a period of time (in some embodiments, the period of time being from 0.5 to 24 hr) to reach maximum crystallization yield. The crystals may be separated from the mother liquor for example by filtration or centrifugation. The crystal cake may be washed with a liquid (in some embodiments, the liquid being the crystallization solvent), and, optionally, dried to obtain crystals with high purity.

When using boiling crystallization, the temperature and the supersaturation gradient between the heat carrier surface and the crystallization mass can be useful in that it fosters the growth of small crystals and can be used to avoid the formation of new crystal nuclei. The rate of crystallization tends to be high because the temperature is suitable, and the viscosity of the mother liquor is low, i.e., mass and heat transport are efficient because of boiling. Boiling crystallization tends to be advantageous for controlling crystal size, as well as achieving yield and crystal quality. The crystals can be separated by, for example, centrifugation.

In some embodiments, the L-fucose crystallization is carried out from a solution having an L-fucose purity of more than 60% on DS. In some embodiments, the crystallization is carried out using cooling crystallization or precipitation crystallization.

In some embodiments, the L-fucose crystallization is carried out from a solution having an L-fucose purity of more than 70% on DS. This embodiment may be carried out by cooling crystallization, by boiling crystallization or by combined boiling-and-cooling crystallization.

In some embodiments, L-fucose crystals having an L-fucose content of greater than 98% on DS (or, alternatively, greater than 99% on DS; or, alternatively, greater than 99.5% on DS) and a low galactose content are obtained by one crystallization step (i.e., single-stage crystallization) from a solution having L-fucose content greater than 65% on DS without using dissolving and recrystallization steps. Single-stage crystallization may comprise boiling and cooling steps, but with no recrystallization step.

In some embodiments, the L-fucose crystals are washed. In some embodiments, the washing is done in connection with the crystal separation from the mother liquor. Additional washing can be done by mixing washing solvent and the crystal cake and then separating crystals. The washing solvent can be, for example, water or alcohol. In some embodiments, this provides L-fucose crystals with a purity of more than 99%.

In some embodiments, the crystallization of L-fucose comprises a single-stage crystallization. In some embodiments, the crystallization of L-fucose comprises boiling crystallization, optionally combined with cooling crystallization. In some embodiments, the crystallization is carried out on a solution having an L-fucose purity of more than 70% on DS. In some embodiments, the crystallization provides crystalline L-fucose having a purity of more than 99.5% on DS and an L-fucose yield of more than 50%. In some embodiments, the crystallization comprises washing the crystals obtained from the crystallization.

In some embodiments, the crystallization is carried out by evaporating the a solution enriched in L-fucose obtained from a chromatographic fractionation or nanofiltration to an appropriate dry substance content (e.g., to an RDS of from about 60 to 90%), depending on the solubility and composition of the liquid. The solution may be seeded with seed crystals of L-fucose. The seeds, if used, may, for example, be pulverized crystals in a dry form or suspended in a crystallization solvent, which may be water, an alcohol (e.g., ethanol), or a mixture thereof. In some embodiments, the crystallization solvent is water. Seed crystals can be made by various processes, including, for example, those discussed in this specification. In some embodiments, the dry seeds are milled to get smaller particle size. The desired amount of seed crystals may depend on, for example, the size of the seed crystals. In some embodiments, crystallization is initiated without adding L-fucose seed crystals to the supersaturated solution. In some such embodiment, for example, seeding is effected using spontaneous seeding. L-fucose seed crystals used herein can be prepared according to, for example, a process discussed in European Patent EP1664352 (incorporated by reference into this specification) or other known processes.

In general, initiation of crystallization (e.g., addition of seed crystals) is carried out when a suitable supersaturation has been achieved. In some embodiments, initiation of crystallization (e.g., addition of seed crystals) is carried out when the L-fucose supersaturation is greater than about 1.0. In some embodiments, initiation of crystallization (e.g., addition of seed crystals) is carried out when the L-fucose supersaturation is from about 1.1 to about 1.8. In some embodiments, initiation of crystallization (e.g., addition of seed crystals) is carried out when the L-fucose supersaturation is from about 1.1 to about 1.5. In some embodiments, initiation of crystallization (e.g., addition of seed crystals) is carried out when the L-fucose supersaturation is from about 1.2 to about 1.4.

In some embodiments, initiation of crystallization (e.g., addition of seed crystals) is carried out when the dry solids content of the syrup is at least about 60% (by weight). In some embodiments, initiation of crystallization (e.g., addition of seed crystals) is carried out when the dry solids content of the syrup is at least about 70% (by weight). In some embodiments, initiation of crystallization (e.g., addition of seed crystals) is carried out when the dry solids content of the syrup is at least about 80% (by weight). In some embodiments, initiation of crystallization (e.g., addition of seed crystals) is carried out when the dry solids content of the syrup is from about 60 to about 90% (by weight). In some embodiments, initiation of crystallization (e.g., addition of seed crystals) is carried out when the dry solids content of the syrup is from about 70 to about 90% (by weight). In some embodiments, initiation of crystallization (e.g., addition of seed crystals) is carried out when the dry solids content of the syrup is from about 80 to about 90% (by weight). In some embodiments, initiation of crystallization (e.g., addition of seed crystals) is carried out when the dry solids content of the syrup is from about 80 to about 88% (by weight).

In some embodiments, the evaporation is continued after seeding, if the crystal growth potential and viscosity allows. After evaporation, the crystallization mass may be subjected to cooling with simultaneous mixing, until the crystal content and viscosity is optimal for separation of crystals. The crystallization mass is typically cooled to a temperature of from 10 to 50° C. The crystallization mass may then be mixed at the final temperature for a period of time (in some embodiments, the time being from 0.5 hr to 24 hr) to reach the maximum crystallization yield, followed by crystal separation by, for example, filtering or centrifuging. In some embodiments, the crystals are washed. This washing is may be done in connection with the crystal separation from the mother liquor. Additional washing may be done by mixing washing solvent and crystal cake and separating crystals thereafter. In some embodiments, the washing solvent is water or alcohol.

In some embodiments, recrystallization is performed one or more times to increase L-fucose purity. Recrystallization may be carried out by, for example, dissolving the L-fucose crystals in water (typically deionized water), bringing the resulting solution to a supersaturated state with respect to L-fucose (via, for example, evaporation), seeding and crystallizing using, for example, the crystallization-by-cooling process described above.

In some embodiments, yield is increased by performing crystallization of the mother liquor produced by the initial crystallization. Such a crystallization may be carried out by, for example, bringing the mother liquor to a supersaturated state with respect to L-fucose (via, for example, evaporation), seeding and crystallizing using, for example, the crystallization-by-cooling process described above.

The crystallization described in this specification does not require an organic solvent to be present in the solution. The absence of an organic solvent can be advantageous. In particular, crystalline L-fucose, which has been produced without adding any organic solvent in crystallization step(s), will be essentially (or completely) free of any organic solvent.

In some embodiments, no alcohol (e.g., methanol, ethanol, etc.) is added to the solution from which the L-fucose is crystallized. In some embodiments, no alcohol is added while the solution is being brought to supersaturation with respect to L-fucose. In some embodiments, no alcohol is added while evaporation is being used to bring the solution to supersaturation with respect to L-fucose. In some embodiments, no alcohol is added while L-fucose crystallization is occurring.

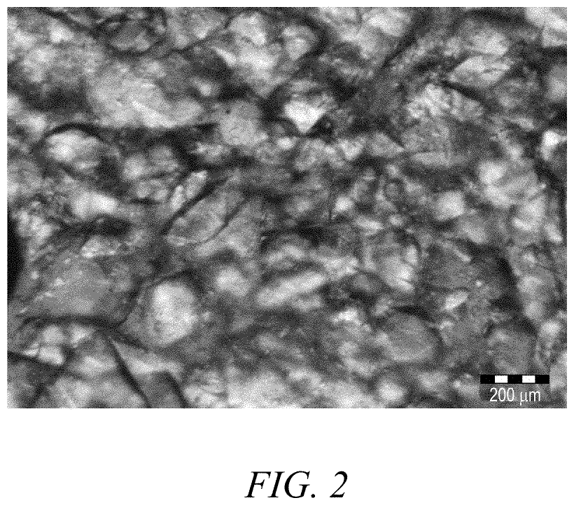

It has been found that L-fucose crystals having essentially cubic crystal habit can be produced using a process of this specification. Essentially cubic crystal habit is crystalline L-fucose where the ratio of the largest and second largest dimension is from 1 to 2. The essentially cubic crystal habit is illustrated in FIG. 2.

The crystalline L-fucose may generally be used as an ingredient to make, for example, dietary supplements, infant nutritional compositions, pharmaceuticals and cosmetics.

### ILLUSTRATIVE EMBODIMENTS

In some embodiments, the selective hydrolysis is performed by adjusting the pH of the starting solution with sulphuric acid to a pH of from 1.0 to 2.0, and keeping the solution at a temperature of from 85 to 96° C. for from 2 to 4 hr. The hydrolysate is cooled and the pH is increased to greater than 2.0 with sodium hydroxide. The neutralized hydrolysate is subjected to a chromatographic separation, which is conducted using strong acid cation resin in Na+-ion form. The recovered L-fucose fraction is crystallized using cooling crystallization at a high temperature of from 75 to 40° C. with water as a solvent.

In some embodiments, the selective hydrolysis is performed by adjusting the pH of the starting solution with sulfuric acid to a pH of from 1.0 to 2.0, and keeping the solution at a temperature of from 85 to 96° C. for from 2 to 4 hr. The hydrolysate is cooled and the pH is increased to a pH of greater than 2.0 with sodium hydroxide. The neutralized hydrolysate is subjected to a chromatographic separation, which is conducted using strong acid cation resin in Na+-ion form. The recovered L-fucose fraction is crystallized using combination of boiling and cooling crystallization at high temperature of from 75 to 40° C. using water as a solvent.

In some embodiments, the selective hydrolysis is performed by adjusting the pH of the starting solution with sulfuric acid to a pH of from 1.0 to 2.0 and keeping the solution at temperature of from 85 to 96° C. for from 2 to 4 hr. The hydrolysate is cooled, and the pH is increased to a pH of greater than 2.0 with sodium hydroxide. The neutralized hydrolysate is subjected to a nanofiltration, and the permeate is further subjected to a chromatographic separation, which is conducted using strong acid cation resin in Na+-ion form. The recovered L-fucose fraction is crystallized using cooling crystallization at a high temperature of from 75 to 40° C. using water as a solvent.

In some embodiments, the selective hydrolysis is performed by adjusting the pH of the starting solution with sulfuric acid to a pH of from 1.0 to 2.0, and keeping the solution at a temperature of from 85 to 96° C. for from 2 to 4 hr. The hydrolysate is cooled, and the pH is increased to a pH of greater than 2.0 with sodium hydroxide. The neutralized hydrolysate is subjected to a nanofiltration, and the permeate is further subjected to chromatographic separation, which is conducted using strong acid cation resin in Na+-ion form. The recovered L-fucose fraction is crystallized using combination of boiling and cooling crystallization at a high temperature of from 75 to 40° C. using water as a solvent.

In some embodiments, the selective hydrolysis is effected with an enzyme at a temperature 40° C. for 24 hr. The hydrolysate is subjected to a chromatographic separation, which is conducted using strong acid cation resin in Na+-ion form. The recovered L-fucose fraction is crystallized using cooling crystallization at a high temperature of from 75 to 40° C. using water as a solvent.

In some embodiments, the selective hydrolysis is effected with an enzyme at a temperature 40° C. for 24 hr. The hydrolysate is subjected to a chromatographic separation, which is conducted using strong acid cation resin in Na+-ion form. The recovered L-fucose fraction is crystallized using a combination of boiling and cooling crystallization at a high temperature of from 75 to 40° C. using water as a solvent.

In some embodiments, the selective hydrolysis is effected with an enzyme at temperature 40° C. for 24 hr. The hydrolysate is subjected to a nanofiltration, which is conducted at a temperature of from 50 to 60° C. The recovered permeate rich in L-fucose is crystallized using cooling crystallization at high temperature of from 75 to 40° C. with water as a solvent.

In some embodiments, the selective hydrolysis is effected with an enzyme at a temperature of from 40° C. for 24 hr. The hydrolysate is subjected to a nanofiltration which is conducted at a temperature of from 50 to 60° C. The recovered permeate rich in L-fucose is crystallized using combination of boiling and cooling crystallization at high temperature of from 75 to 40° C. with water as a solvent.

In some embodiments, the selective hydrolysis is effected with an enzyme at a temperature of 40° C. for 24 hr. The hydrolysate is subjected to a nanofiltration, which is conducted at a temperature of from 50 to 60° C. The nanofiltration permeate is further hydrolyzed with an enzyme to hydrolyze lactose to galactose and glucose. The hydrolysate rich in L-fucose is crystallized using cooling crystallization at high temperature of from 75 to 40° C. with water as a solvent.

In some embodiments, the selective hydrolysis is effected with an enzyme at a temperature of 40° C. for 24 hr. The hydrolysate is subjected to nanofiltration, which is conducted at a temperature of from 50 to 60° C. The nanofiltration permeate is further hydrolyzed with an enzyme to hydrolyze lactose to galactose and glucose. The hydrolysate rich in L-fucose is crystallized using combination of boiling and cooling crystallization at a high temperature of from 75 to 40° C. with water as a solvent.

In some embodiments, the selective hydrolysis is performed using a column filled with strong acid cation ion exchange resin in H⁺-ion form at a temperature of 70° C. with a flow rate of from 0.2 to 2.0 BV/h. The hydrolysate is subjected to a chromatographic separation, which is conducted using strong acid cation resin in Na+-ion form. The recovered L-fucose fraction is crystallized using cooling crystallization at high temperature of from 75 to 40° C. with water as a solvent.

In some embodiments, the selective hydrolysis is performed using column filled with strong acid cation ion exchange resin in H⁺-ion form at a temperature of 70° C. with a flow rate of from 0.2 to 2.0 BV/h. The hydrolysate is subjected to a chromatographic separation, which is conducted using strong acid cation resin in Na+-ion form. The recovered L-fucose fraction is crystallized using combination of boiling and cooling crystallization at high temperature of from 75 to 40° C. with water as a solvent.

The following provides further illustration of various embodiments:

Embodiment 1: A process for producing substantially pure L-fucose, comprising:
i. selective hydrolysis of an HMO, which contains one or more L-fucose moieties in its structure, to yield a mixture comprising principally fucose, lactose, galactose and glucose;
ii. subjecting the mixture to chromatographic separation and/or nanofiltration, and recovering a fraction enriched in L-fucose; and
iii. crystallization of fucose from the recovered fraction to produce substantially pure fucose.

Embodiment 2: A process according to Embodiment 1, wherein the HMO containing one or more L-fucose moieties in its structure is selected from 2′-fucosyllactose (2′-FL), 3-fucosyllactose (3-FL) and/or difucosyllactose (LDFT).

Embodiment 3: A process according to any one preceding embodiment, wherein the starting material is selected from a liquor derived from a fermentation containing 2′-fucosyllactose, crystalline or spray dried 2′-fucosyllactose, purified 2′-fucosyllactose syrup and 2′-fucosyllactose crystallization mother liquor.

Embodiment 4: A process according to any one preceding embodiment, wherein the starting material is a liquor derived from a fermentation containing 3-fucosyllactose, crystalline or spray dried 3-fucosyllactose product, purified 3-fucosyllactose syrup and 3-fucosyllactose crystallization mother liquor.

Embodiment 5: A process according to any one preceding embodiment, wherein the starting material is selected from a liquor derived from a fermentation containing difucosyllactose, crystalline or spray dried difucosyllactose product, purified difucosyllactose syrup and difucosyllactose crystallization mother liquor.

Embodiment 6: A process according to any one preceding embodiment, wherein selective hydrolysis is initiated by an acid or enzyme or is performed using an ion exchange column with a strong acid cation (SAC) ion exchange resin in H⁺-ion form.

Embodiment 7: A process according to any one preceding embodiment, wherein the selective hydrolysis is initiated by an acid selected from mineral acids, organic acids and inorganic acids.

Embodiment 8: A process according to any one preceding embodiment, wherein the inorganic acid is sulfuric acid.

Embodiment 9: A process according to any one preceding embodiment, wherein the selective hydrolysis is initiated by an acid and is performed at a pH of from 1.00 to 2.00; or, alternatively, at a pH of from 1.5 to 1.75.

Embodiment 10: A process according to any one preceding embodiment, wherein the selective hydrolysis initiated by an acid and is performed at a temperature of from 70 to 140° C.; or, alternatively, from 80 to 110° C.; or, alternatively, from 90 to 100° C.; or, alternatively, from 85 to 96° C.

Embodiment 11: A process according to any one preceding embodiment, wherein the selective hydrolysis initiated by an acid, and is carried out with a residence time of from 1 to 24 hr; or, alternatively, from 0.5 to 6 hr; or, alternatively, from 1 to 4 hr; or, alternatively, from 2 to 4 hr.

Embodiment 12: A process according to any one preceding embodiment, wherein the hydrolysate is neutralized by addition of an alkali selected from CaO, MgO, NaOH, KOH, Na₂CO₃ and CaCO₃. In some embodiments, the alkali is NaOH or KOH.

Embodiment 13: A process according to any one preceding embodiment, wherein the selective hydrolysis is initiated by an enzyme.

Embodiment 14: A process according to any one preceding embodiment, wherein the enzyme has a 1,2-α-L-fucosidase activity or 1,3/4-α-L-fucosidase activity.

Embodiment 15: A process according to any one preceding embodiment, wherein the selective hydrolysis is performed using strong acid cation ion exchange resin in H⁺-ion form.

Embodiment 16: A process according to any one preceding embodiment, wherein the selective hydrolysis of step (i) provides an aqueous hydrolysate having a fucose yield of more than 50% of the theoretical fucose yield from the 2′-FL, 3-FL and/or LDFT.

Embodiment 17: A process according to any one preceding embodiment, wherein the selective hydrolysis of step (i) provides an aqueous hydrolysate having a fucose yield of more than 60% of the theoretical fucose yield from the 2′-FL, 3-FL and/or LDFT.

Embodiment 18: A process according to any one preceding embodiment, wherein the selective hydrolysis of step (i) provides an aqueous hydrolysate having a fucose yield of more than 70% of the theoretical fucose yield from the 2′-FL, 3-FL and/or LDFT.

Embodiment 19: A process according to any one preceding embodiment, wherein the selective hydrolysis of step (i) provides an aqueous hydrolysate having a fucose yield of more than 80% of the theoretical fucose yield from the 2′-FL, 3-FL and/or LDFT.

Embodiment 20: A process according to any one preceding embodiment, wherein the selective hydrolysis of step (i) provides an aqueous hydrolysate where the yields of galactose and glucose are less than 20% based on the lactose that could potentially be formed by the hydrolysis (and, in turn, hydrolyzed into galactose and glucose).

Embodiment 21: A process according to any one preceding embodiment, wherein the selective hydrolysis of step (i) provides an aqueous hydrolysate where the yields of galactose and glucose are less than 15% based on the lactose that could potentially be formed by the hydrolysis.

Embodiment 22: A process according to any one preceding embodiment, wherein the selective hydrolysis of step (i) provides an aqueous hydrolysate where the yields of galactose and glucose are less than 12% based on the lactose that could potentially be formed by the hydrolysis.

Embodiment 23: A process according to any one preceding embodiment, wherein the selective hydrolysis of step (i) provides an aqueous hydrolysate where the yields of galactose and glucose are less than 10% based on the lactose that could potentially be formed by the hydrolysis.

Embodiment 24: A process according to any one preceding embodiment, wherein the hydrolysis results in a L-fucose yield of more than 75% of the theoretical yield based on the starting material, and the galactose and glucose yields are less than 12% based on the lactose that could potentially be formed by the hydrolysis.

Embodiment 25: A process according to any one preceding embodiment, wherein the hydrolysis results in a L-fucose yield of more than 80% of the theoretical yield based on the starting material, and the galactose and glucose yields are less than 10% based on the lactose that could potentially be formed by the hydrolysis.

Embodiment 26: A process according to any one preceding embodiment, wherein the hydrolysis of step (i) provides an aqueous hydrolysate where the content of fucose is more than 10% on DS; or, alternatively, more than 15% on DS; or, alternatively, more than 20% on DS; or, alternatively, more than 25% on DS.

Embodiment 27: A process according to any one preceding embodiment, wherein the chromatographic separation is performed using a resin selected from strong acid cation (SAC) resins and weak acid cation (WAC) resins.

Embodiment 28: A process according to any one preceding embodiment, wherein the chromatographic separation is performed using strong acid cation resin in Na⁺-ion form.

Embodiment 29: A process according to any one preceding embodiment, wherein chromatographic separation is performed using weak acid cation resin in H⁺-ion form.

Embodiment 30: A process according to any one preceding embodiment, wherein the chromatographic separation comprises simulated moving bed separation or batch separation.

Embodiment 31: A process according to any one preceding embodiment, wherein the purity of the fucose fraction recovered from the chromatographic separation is more than 60% on DS; or, alternatively, more than 70% on DS; or, alternatively, more than 80% on DS; or, alternatively, more than 90% on DS.

Embodiment 32: A process according to any one preceding embodiment, wherein the chromatographic separation results in a fucose yield of more than 70% (or, alternatively, more than 80%; or, alternatively, at least 90%) obtained, based on the fucose present in the hydrolysate feed.

Embodiment 33: A process according to any one preceding embodiment, wherein fractionation of hydrolysate is carried out by nanofiltration.

Embodiment 34: A process according to any one preceding embodiment, wherein the purity of the permeate containing fucose recovered from the nanofiltration is more than 50% on DS; or, alternatively, more than 60% on DS; or, alternatively, more than 70% on DS.

Embodiment 35: A process according to any one preceding embodiment, wherein the fucose yield from the nanofiltration is more than 50% (or, alternatively, more than 70%; or, alternatively, at least 90%) based on the fucose present in the hydrolysate feed.

Embodiment 36: A process according to any one preceding embodiment, wherein the permeate collected from the nanofiltration is subjected to a subsequent enzymatic hydrolysis initiated by lactase.

Embodiment 37: A process according to any one preceding embodiment, wherein nanofiltration is performed before chromatographic separation.

Embodiment 38: A process according to any one preceding embodiment, wherein one or more fractions enriched in fucose is/are subjected to crystallization.

Embodiment 39: A process according to any one preceding embodiment, wherein crystallization is carried out using boiling and/or cooling crystallization.

Embodiment 40: A process according to any one preceding embodiment, wherein the fucose content in the crystallization feed is more than 70% on DS; or, alternatively, more than 80% on DS; or, alternatively, more than 90% on DS; or, alternatively, more than 95% on DS

Embodiment 41: A process according to any one preceding embodiment, wherein fucose is crystallized from a solvent selected from water, an alcohol (in some embodiments, ethanol), and a mixture of water and an alcohol.

Embodiment 42: A process according to any one preceding embodiment, wherein the crystallization solvent is water.

Embodiment 43: A process according to any one preceding embodiment, wherein no organic solvent is added to the crystallization feed before concentration of the solution to a supersaturated state.

Embodiment 44: A process according to any one preceding embodiment, wherein no organic solvent is added to the crystallization feed during concentration of the solution to a supersaturated state.

Embodiment 45: A process according to any one preceding embodiment, wherein the crystallization is carried out at a temperature of from 10 to 80° C.

Embodiment 46: A process according to any one preceding embodiment, wherein the process provides crystalline fucose with a purity of more than 90% on DS; or, alternatively, more than 95% on DS; or, alternatively, more than 99% on DS.

Embodiment 47: A process according to any one preceding embodiment, wherein the process comprises washing the crystals obtained from the crystallization.

Embodiment 48: A process according to any one preceding embodiment, wherein the process comprises recrystallization of fucose.

Embodiment 49: Crystalline L-fucose product obtained from a process of any one of the preceding embodiments.

Embodiment 50: Crystalline L-fucose according to Embodiment 49, having a purity of at least 99.0% on DS, and is essentially free from organic solvents.

Embodiment 51: Crystalline L-fucose according to any one of Embodiments 49 or 50 having melting point of at least 140° C.

Embodiment 52: Crystalline L-fucose according to any one of Embodiments 49 to 51 having essentially cubic crystal habit.

Embodiment 53: Use of L-fucose of any one of the preceding embodiments in a pharmaceutical.

Embodiment 54: Use of L-fucose of any one of the preceding embodiments in infant nutrition.

Embodiment 55: Use of L-fucose of any one of the preceding embodiments in food.

Embodiment 56: Use of L-fucose of any one of the preceding embodiments in a feed.

### EXAMPLES

The following examples are merely illustrative, and not limiting to the remainder of this specification in any way.

In the examples, unless otherwise mentioned, fucose refers to L-fucose, and references to other sugars (such as galactose) refer to the sugar in D-form.

HPLC analysis process has been used to determine the compositions of the feed liquors, hydrolysates, L-fucose fractions and crystallization products. Peak area purity or concentration obtained using HPLC has been used.

Dry solids (DS) were measured by Karl Fischer titration or refractive index process.

Colour was determined under the International Commission for Uniform Process of Sugar Analysis (“ICUMSA”) sugar colour grading system

Melting point was measured with a 1° C./min heating rate using the European Pharmacopoeia capillary melting point process.

HPLC refers to high performance liquid chromatography.

### Example 1

### Selective Hydrolysis of 2′-FL Crystallization Mother Liquor with Thermal Degradation and Mild Acid Condition

2′-FL crystallization mother liquor was obtained from crystallization of purified 2′-FL syrup. Selective hydrolysis using thermal degradation was done in 50 ml Schott flask with 4 g liquid. Mild acid hydrolysis was carried out in a 2-liter Schott flask with close to 1700 g liquid. Concentration for hydrolysis was adjusted to 45% brix in both tests. For thermal the degradation test, the pH of the feed was 5.1, and in the mild acid test, the pH was adjusted to 3 with 0.5M H₂SO₄. Both tests were carried out in an oven for 14 days at 85° C. Crystallization mother liquors were composed as set forth below in Table 1-1, whereby the HPLC analyses are given on peak area-% basis.

TABLE 1-1

| Composition of 2′-FL | | |
|---|---|---|
| Crystallization mother liquors | Thermal degradation | Mild acid |
| L-fucose, % | 0.3 | 0.1 |
| Galactose + Glucose, % | 1.6 | 0.1 |
| Lactose, % | 0.3 | 1.7 |
| 2′fucosyllactose, % | 80.2 | 70.6 |
| Difucosyllactose, % | 13.2 | 26.2 |
| Others, % | 4.4 | 1.3 |

During the 14-day thermal degradation, the pH decreased to 3.4. The mild acid hydrolysate pH was 2.9 after 14 days. The fucose yield out of theoretical value was 89% and 95% in thermal degradation and mild acid hydrolysis, respectively. Glucose and galactose yield from potential lactose were 17% and 19% in thermal degradation and mild acid hydrolysis, respectively. Hydrolysates were composed as set forth below in Table 1-2, whereby the HPLC analyses are given on peak area-% basis.

TABLE 1-2

| | Thermal degradation | Mild acid |
|---|---|---|
| Composition of hydrolysates | (%) | (%) |
| L-fucose | 30.1 | 28.6 |
| Galactose + Glucose | 11.6 | 8.9 |
| Lactose | 44.2 | 43.7 |
| 2′fucosyllactose | 7.9 | 4.4 |
| Difucosyllactose | 0.3 | 0.0 |
| Others | 5.9 | 14.4 |

### Example 2

### Acid Hydrolysis of 2′-FL Crystallization Mother Liquor in Different Conditions

2′-FL crystallization mother liquor was obtained from crystallization of purified 2′-FL syrup. Acid hydrolysis was carried out in 100 ml Schott flasks in 94° C. water bath with 50 g liquid. Concentration for hydrolysis was adjusted to 44-45% brix and the pH was adjusted to 1, 1.5, 1.75 or 2.0 with 0.5M H₂SO₄. The duration of hydrolysis was 2 or 4 hr. Crystallization mother liquor was composed as set forth in Table 2-1, whereby the HPLC analyses are given on peak area-% basis.

TABLE 2-1

| Composition of 2′-FL Crystallization mother liquor | % |
|---|---|
| L-fucose | 0.0 |
| Galactose + Glucose | 0.0 |
| Lactose | 21.2 |
| 2′fucosyllactose | 65.0 |
| Difucosyllactose | 11.3 |
| Others | 2.5 |

The fucose yield out of theoretical value was from 39 to 85%, depending on the conditions. Glucose and galactose yield from potential lactose were 1% and 58% depending on the concentration. Hydrolysates were composed as set forth below in Table 2-2, whereby the HPLC analyses are given on peak area-% basis.

TABLE 2-2

| | Duration, hr | | | | | | | |
|---|---|---|---|---|---|---|---|---|
| | 2 h | | | | 4 h | | | |
| pH | 1 | 1.5 | 1.75 | 2 | 1 | 1.5 | 1.75 | 2 |
| L-fucose, % | 22.9 | 22.3 | 15.9 | 9.9 | 23.4 | 22.9 | 21.1 | 15.5 |
| Galactose + | 29.9 | 8.4 | 1.1 | 1.8 | 47.0 | 16.3 | 2.9 | 3.3 |
| Glucose, % | | | | | | | | |
| Lactose, % | 35.2 | 54.6 | 47.6 | 37.1 | 18.7 | 49.9 | 54.4 | 46.5 |
| 2′fucosyl- | 0.0 | 6.0 | 26.0 | 41.1 | 0.0 | 0.8 | 8.3 | 25.0 |
| lactose, % | | | | | | | | |
| Difucosyl- | 0.0 | 0.0 | 1.6 | 3.9 | 0.0 | 0.0 | 0.0 | 1.5 |
| lactose, % | | | | | | | | |
| Others, % | 11.9 | 8.5 | 7.8 | 6.2 | 10.9 | 10.1 | 13.3 | 8.2 |
| % of Galactose | 38.4 | 11.0 | 1.4 | 2.5 | 58.0 | 20.7 | 3.8 | 4.5 |
| and glucose | | | | | | | | |
| formed on | | | | | | | | |
| potential | | | | | | | | |
| lactose | | | | | | | | |
| % fucose | 84.4 | 83.7 | 56.4 | 38.7 | 82.9 | 83.6 | 75.3 | 59.9 |
| yield out of | | | | | | | | |
| theoretical | | | | | | | | |

Selective hydrolysis at a moderately acidic pH of from 1.5 to 2 results in reasonable fucose yield, while minimizing the hydrolysis of lactose to galactose and glucose.

### Example 3

### Acid Hydrolysis of 2′-FL Crystallization Mother Liquor

2′-FL crystallization mother liquor was obtained from crystallization of purified 2′-FL syrup. Acid hydrolysis was carried out in 8-liter jacketed steel vessel with mixing. 8.2 kg of crystallization mother liquor was fed into the vessel, corresponding to 3.6 kg dry substance (DS). The material was first heated with steam to 85° C. Then 14.4 g of 2.5M H₂SO₄ was added to adjust the pH to 1.5. Heating was continued to reach 94° C. and then kept at a temperature of from 93 to 95° C. for 2 hr. The hydrolysate was then cooled to 37° C., and the pH was increased to 2.2 with 1 M and 30% NaOH.

Crystallization mother liquor was composed as set forth in Table 3-1, whereby the HPLC analyses are given on peak area-% basis.

TABLE 3-1

| Composition of 2′-FL Crystallization mother liquor | % |
|---|---|
| L-fucose | 0.0 |
| Galactose + Glucose | 0.0 |
| Lactose | 21.2 |
| 2′fucosyllactose | 65.0 |
| Difucosyllactose | 11.3 |
| Others | 2.5 |

Hydrolysate was composed as set forth in Table 3-2, whereby the HPLC analyses are given on peak area-% basis.

TABLE 3-2

| Composition of hydrolysate | % |
|---|---|
| L-fucose | 22.4 |
| Galactose + Glucose | 8.7 |
| Lactose | 54.8 |
| 2′fucosyllactose | 5.9 |
| Difucosyllactose | 0.0 |
| Others | 8.3 |

The fucose yield out of theoretical value was 80.0% and galactose and glucose yield out of potential lactose was 11.4%.

### Example 4

### Acid Hydrolysis of High Purity 2′-FL Product

Acid hydrolysis was carried out in 8-liter jacketed steel vessel with mixing. Wet 2′-FL crystals, corresponding to 1.9 kg of dry substance (DS), were dissolved in 2.3 kg ion exchanged water. The material was first heated with steam to 85-90° C. Then the pH was adjusted to 1.4-1.5 with 2.5M H₂SO₄. Heating was continued to reach 95° C. and then kept at a temperature of from 88 to 96° C. for 100 min. The hydrolysate was then cooled to 47° C. and the pH was increased to 2.3 with 30% NaOH.

Feed crystals was composed as set forth in Table 4-1, whereby the HPLC analyses are given on peak area-% basis.

TABLE 4-1

| Composition of 2′-FL feed crystals | % |
|---|---|
| L-fucose | 0.0 |
| Galactose + Glucose | 0.0 |
| Lactose | 0.4 |
| 2′fucosyllactose | 99.2 |
| Difucosyllactose | 0.0 |
| Others | 0.4 |

Hydrolysate was composed as set forth in Table 4-2, whereby the HPLC analyses are given on peak area-% basis.

TABLE 4-2

| Composition of hydrolysate | % |
|---|---|
| L-fucose, % | 25.6 |
| Galactose + Glucose, % | 6.2 |
| Lactose, % | 53.0 |
| 2′fucosyllactose, % | 10.8 |
| Difucosyllactose, % | 0.0 |
| Others, % | 4.4 |

The fucose yield out of theoretical value was 80.2% and galactose and glucose yield out of potential lactose was 8.4%.

### Example 5

### Enzymatic Hydrolysis of 2′-FL Crystallization Mother Liquor

2′-FL crystallization mother liquor was obtained from crystallization of purified 2′-FL syrup. Hydrolysis was carried out in 100 ml Schott flask in 40° C. water bath with 50 g liquid. Concentration for hydrolysis was adjusted to 44-45% brix and the pH was measured to be 5.5. 100 μl of Enzyme (1,2-alpha-L-fucosidase, EC 3.2.1.63) corresponding about 100 IU (6.4 IU/g HMO) was added. Duration of hydrolysis was 24 hr. Crystallization mother liquor was composed as set forth in Table 5-1, whereby the HPLC analyses are given on peak area-% basis.

TABLE 5-1

| Composition of 2′-FL Crystallization mother liquor | % |
|---|---|
| L-fucose | 0.0 |
| Galactose + Glucose | 0.0 |
| Lactose | 21.2 |
| 2′fucosyllactose | 65.0 |
| Difucosyllactose | 11.3 |
| Others | 2.5 |

The fucose yield out of theoretical value was about 91% after 24 hr. There was no hydrolysis of lactose to galactose and glucose. Hydrolysates were composed as set forth in Table 5-2, whereby the HPLC analyses are given on peak area-% basis.

TABLE 5-2

| Duration, hr | 2 | 4 | 24 |
|---|---|---|---|
| L-fucose, % | 8.8 | 13.4 | 22.2 |
| Galactose + Glucose, % | 0.0 | 0.0 | 0.0 |
| Lactose, % | 37.4 | 45.7 | 61.0 |
| 2′fucosyllactose, % | 39.9 | 26.0 | 1.2 |
| Difucosyllactose, % | 9.8 | 9.4 | 6.2 |
| Others, % | 4.0 | 5.5 | 9.4 |

### Example 6

### Enzymatic Hydrolysis of 2′-FL Crystallization Mother Liquor

2′-FL crystallization mother liquor was obtained from crystallization of purified 2′-FL syrup. Hydrolysis was carried out for 4 kg of feed liquor in concentration 45 g/100 g in an 8-liter vessel with mixing. The pH was adjusted with dilute NaOH to be 5.5, and 8 ml of Enzyme (1,2-alpha-L-fucosidase, EC 3.2.1.63) was added. Hydrolysis was continued for 24 hr in constant temperature of 40° C. Crystallization mother liquor was composed as set forth in Table 6-1, whereby the HPLC analyses are given on peak area-% basis.

TABLE 6-1

| Composition of hydrolyzation feed | % |
|---|---|
| L-fucose | 0.0 |
| Galactose + Glucose | 0.0 |
| Lactose | 15.0 |
| 2′fucosyllactose | 66.0 |
| Difucosyllactose | 19.0 |
| Others | 0.0 |

The fucose yield out of theoretical value was about 91% after 24 hr. There was no hydrolysis of lactose to galactose and glucose. The hydrolysate was composed as set forth in Table 6-2, whereby the HPLC analyses are given on peak area-% basis.

TABLE 6-2

| Composition of hydrolysate | % |
|---|---|
| L-fucose | 26.4 |
| Galactose + Glucose | 0.0 |
| Lactose | 57.0 |
| 2′fucosyllactose | 1.2 |
| Difucosyllactose | 9.5 |
| Others | 5.9 |

### Example 7

### Acid Hydrolysis of Purified 3-FL Syrup

Purified 3-FL syrup was used as feed. Acid hydrolysis was carried out in 100 ml Schott flasks in 94° C. water bath with 50 g liquid. Concentration for hydrolysis was adjusted to 49.3 g/100 g and the pH was adjusted to 1.5, 1.75 or 2.0 with 0.5M H₂SO₄. Duration of hydrolysis was 2 or 4 hr. 3-FL syrup was composed as set forth in Table 7-1, whereby the HPLC analyses are given on peak area-% basis.

TABLE 7-1

| Composition of purified 3-FL syrup | % |
|---|---|
| L-fucose | 2.5 |
| Galactose + Glucose | 3.0 |
| Lactose | 3.5 |
| 3-fucosyllactose | 88.2 |
| Difucosyllactose | 0.0 |
| Others | 2.8 |

The fucose yield out of theoretical value was from 55 to 90%, depending on the conditions and duration. Glucose and galactose yield from potential lactose were 4% and 9% depending on the concentration. Hydrolysates were composed as set forth in Table 7-2, whereby the HPLC analyses are given on peak area-% basis.

TABLE 7-2

| | Duration, h | | | | | |
|---|---|---|---|---|---|---|
| | 2 h | | | 4 h | | |
| pH | 1.5 | 1.75 | 2 | 1.5 | 1.75 | 2 |
| L-fucose, % | 26.2 | 24.5 | 17.1 | 25.3 | 25.7 | 22.1 |
| Galactose + Glucose, % | 4.3 | 3.7 | 2.9 | 6.3 | 4.2 | 3.4 |
| Lactose, % | 55.6 | 53.0 | 36.8 | 48.6 | 54.8 | 48.1 |
| 3-fucosyllactose, % | 2.2 | 9.5 | 36.6 | 0.0 | 1.9 | 17.8 |
| Difucosyllactose, % | 0.0 | 0.0 | 0.0 | 0.0 | 0.0 | 0.0 |
| Others, % | 10.9 | 8.7 | 6.6 | 18.6 | 12.4 | 8.0 |
| % of Galactose and glucose | 5.6 | 5.1 | 4.0 | 8.4 | 5.8 | 4.7 |
| formed on potential lactose | | | | | | |
| % fucose yield out of | 80.8 | 77.9 | 55.4 | 92.7 | 82.6 | 70.9 |
| theoretical | | | | | | |

### Example 8

### Purified 3-FL Syrup Hydrolysis with H+ Form Strong Acid Cation (SAC) Ion Exchange Resin

Purified 3-FL syrup was used as feed. 200 ml of Dowex 88 strong acid cation (SAC) ion exchange resin was regenerated with 800 ml of 5% sulphuric acid solution to get resin into H+ form. Purified 3-FL syrup was diluted with deionized water to 39.8% Brix feed solution into SAC (H+) column. SAC (H+) column temperature was adjusted to 70° C. and feed into column was fed with 50 ml/h (0.25 BV/h) flow rate. Product from SAC (H+) column was collected in 60 min fractions, 50 ml each, and analyzed. Purified 3-FL syrup was composed as set forth in Table 8-1, whereby the HPLC analyses are given on peak area-% basis.

TABLE 8-1

| Composition of purified 3-FL syrup | |
|---|---|
| pH | 6.53 |
| L-fucose, % | 2.3 |
| Galactose + Glucose, % | 2.5 |
| Lactose, % | 3.3 |
| 3-fucosyllactose, % | 90.3 |
| Difucosyllactose, % | 0.0 |
| Others, % | 1.6 |

Hydrolysates out from SAC (H+) column were composed as set forth in Table 8-2, whereby the HPLC analyses are given on peak area-% basis. 73.5% L-fucose yield of theoretical maximum with this feed solution was achieved in 4-5 hour fraction.

TABLE 8-2

| | SAC (H+) product | | | | |
|---|---|---|---|---|---|
| | fraction collected, h | | | | |
| | 0-1 | 1-2 | 2-3 | 3-4 | 4-5 |
| pH | 3.30 | 3.19 | 3.17 | 3.15 | 3.14 |
| L-fucose, % | 16.5 | 19.6 | 21.6 | 23.1 | 24.1 |
| Galactose + Glucose, % | 4.0 | 5.4 | 6.7 | 8.5 | 9.1 |
| Lactose, % | 35.4 | 40.8 | 44.9 | 47.0 | 48.0 |
| 3-fucosyllactose, % | 41.5 | 31.2 | 23.7 | 18.7 | 16.0 |
| Difucosyllactose, % | 0.0 | 0.0 | 0.0 | 0.0 | 0.0 |
| Others, % | 2.6 | 3.0 | 3.1 | 2.7 | 2.8 |

### Example 9

### 2′FL Crystallization Mother Liquor Hydrolysis with H+ Form Strong Acid Cation (SAC) Ion Exchange Resin

2′-FL crystallization mother liquor was obtained from crystallization of 2′-FL mother liquor. 200 ml of Dowex 88 strong acid cation (SAC) ion exchange resin was regenerated with 800 ml of 5% sulphuric acid solution to get resin into H+ form. 2′-FL crystallization mother liquor was diluted with deionized water to 40.8% Brix feed solution into SAC (H+) column. SAC (H+) column temperature was adjusted to 80° C. and feed into column was fed with 40 ml/h (0.2 BV/h) flow rate. Product from SAC (H+) column was collected in 60 min fractions, 40 ml each, and analyzed. Crystallization mother liquor was composed as set forth in Table 9-1, whereby the HPLC analyses are given on peak area-% basis.

TABLE 9-1

| Composition of 2′-FL Crystallization mother liquor | |
|---|---|
| pH | 4.20 |
| L-fucose, % | 0.5 |
| Galactose + Glucose, % | 0.4 |
| Lactose, % | 18.0 |
| 2′fucosyllactose, % | 55.5 |
| Difucosyllactose, % | 19.5 |
| Others, % | 6.1 |

Hydrolysates out from SAC (H+) column were composed as set forth in Table 9-2, whereby the HPLC analyses are given on peak area-% basis. 85.7% L-fucose yield of theoretical maximum with this feed solution was achieved in 3-10 hour fractions average.

TABLE 9-2

| | SAC (H+) product | | | | | |
|---|---|---|---|---|---|---|
| | fraction collected, hr | | | | | |
| | 0-1 | 1-2 | 3-4 | 5-6 | 7-8 | 9-10 |
| pH | 2.84 | 2.82 | 2.80 | 2.73 | 2.65 | 2.58 |
| L-fucose, % | 20.4 | 22.8 | 24.5 | 25.0 | 25.4 | 25.9 |
| Galactose + Glucose, % | 8.5 | 13.6 | 22.9 | 29.4 | 33.7 | 42.8 |
| Lactose, % | 42.6 | 42.4 | 37.0 | 31.9 | 28.4 | 19.9 |
| 2′fucosyllactose, % | 14.5 | 9.1 | 4.0 | 1.9 | 1.0 | 0.6 |
| Difucosyllactose, % | 4.4 | 2.7 | 1.3 | 0.8 | 0.5 | 0.2 |
| Others, % | 9.6 | 9.4 | 10.3 | 11.0 | 11.0 | 10.6 |

### Example 10

### 2′-FL Crystallization Mother Liquor Hydrolysis with H+ Form Strong Acid Cation (SAC) Ion Exchange Resin

2′-FL crystallization mother liquor was obtained from crystallization of purified 2′-FL syrup. 200 ml of Dowex 88 strong acid cation (SAC) ion exchange resin was regenerated with 800 ml of 5% sulphuric acid solution to get resin into H+ form. 2′-FL crystallization mother liquor was diluted with deionized water to 40.7% dry substance feed solution into SAC (H+) column. SAC (H+) column temperature was adjusted to 75° C. and feed into column was fed with 50 ml/h (0.25 BV/h) flow rate. Product from SAC (H+) column was collected in 4 hour fractions for 84 hr (21 bed volumes), 200 ml in each fraction, and analyzed. Crystallization mother liquor was composed as set forth in Table 10-1, whereby the HPLC analyses are given on peak area-% basis.

TABLE 10-1

| Composition of 2′-FL Crystallization mother liquor | |
|---|---|
| pH | 4.16 |
| L-fucose, % | 0.5 |
| Galactose + Glucose, % | 0.5 |
| Lactose, % | 17.5 |
| 2′fucosyllactose, % | 52.4 |
| Difucosyllactose, % | 24.2 |
| Others, % | 4.9 |

Hydrolysates out from SAC (H+) column were composed as set forth in Table 10-2, whereby the HPLC analyses are given on peak area-% basis. 76.0% fucose yield of theoretical maximum with this feed solution was achieved in this test. Galactose and glucose formation was more moderate in this test compared to Example 9. This was achieved by lowering temperature and shortening residence time in the SAC (H+) column.

TABLE 10-2

| | SAC (H+) product | | | | | |
|---|---|---|---|---|---|---|
| | fraction collected, hr | | | | | |
| | 0-4 | 16-20 | 36-40 | 48-52 | 64-68 | 80-84 |
| pH | 2.81 | 2.63 | 2.55 | 2.58 | 2.54 | 2.60 |
| L-fucose, % | 13.8 | 23.1 | 22.5 | 22.6 | 22.6 | 22.5 |
| Galactose + Glucose, % | 4.1 | 10.0 | 9.9 | 9.9 | 9.8 | 10.0 |
| Lactose, % | 33.2 | 42.4 | 41.9 | 41.8 | 41.8 | 41.8 |
| 2′fucosyllactose, % | 28.3 | 10.3 | 10.9 | 10.8 | 11.1 | 11.0 |
| Difucosyllactose, % | 12.1 | 3.8 | 4.2 | 4.1 | 4.2 | 4.1 |
| Others, % | 8.5 | 10.4 | 10.6 | 10.8 | 10.5 | 10.6 |

### Example 11

### Nanofiltration of 2′-FL Crystallization Mother Liquor Mild Acid Hydrolysate

The process equipment included a plate and frame filtration unit (Alfa Laval Labstak M20), feed and diafiltration pump, heat exchanger, heating water bath, 8-liter feed tank as well as inlet and outlet pressure gauges and pressure control valve. The total membrane area was 0.54 m². The membrane installed was Desal 5 DK series (Suez) with an approximate molecular weight cut-off of 150-300 Dalton and 98-99% MgSO₄ retention at 25° C.

As a feed, HMO hydrolysate from a mild acid hydrolysis at a pH of 3 for 14 days was used, and the aim was to separate L-fucose contained therein to the NF permeate while retaining lactose.

5.66 kg of feed was fed to an 8-liter feed tank. The liquor concentration was 14.7 g/100 g and the pH was 3.5. The hydrolysate was composed as set forth in Table 11-1, whereby the HPLC analyses are given on peak area-% basis.

TABLE 11-1

| Composition of Feed | % |
|---|---|
| L-fucose | 28.7 |
| Galactose + Glucose | 9.0 |
| Lactose | 43.5 |
| 2′fucosyllactose | 4.6 |
| Difucosyllactose | 0.0 |
| Others | 14.2 |

The feed was heated to 60° C. and water was used for diafiltration. The filtration pressure was set at 20-30 Bar and concentrate DS concentration controlled to keep flux at greater than 6 kg/m²/h. After batch filtration, a permeate fraction and final concentrate fraction were collected. The result including HPLC analyses on peak area-% basis for the permeate fractions, final concentrate and combined evaporated permeate are set forth in Table 11-2.

TABLE 11-2

| | Permeate | Final concentrate |
|---|---|---|
| mass, kg | 10.5 | 3.0 |
| Dry substance, g/100 g | 3.0 | 16.7 |
| L-fucose, % | 60.1 | 8.7 |
| Galactose + Glucose. % | 17.1 | 4.0 |
| Lactose, % | 16.2 | 62.2 |
| 2′fucosyllactose | 0.9 | 6.5 |
| Difucosyllactose, % | 0.0 | 0.0 |
| Others, % | 5.8 | 18.5 |

The overall L-fucose yield calculated from the permeate fractions was 81.7%.

### Example 12

### Nanofiltration of 2′-FL Crystallization Mother Liquor Acid Hydrolysate

The process equipment included a plate and frame filtration unit (Alfa Laval Labstak M20), feed and diafiltration pump, heat exchanger, heating water bath, 8-liter feed tank as well as inlet and outlet pressure gauges and pressure control valve. The total membrane area was 0.72 m². The membrane installed was Desal 5 DK series (Suez) with an approximate molecular weight cut-off of 150-300 Dalton and 98-99% MgSO₄ retention at 25° C.

As a feed, neutralized HMO hydrolysate from 1.5 pH acid hydrolysis of crystallization mother liquor was used, and the aim was to separate L-fucose contained therein to the NF permeate while retaining lactose.

3.6 kg of feed was fed to an 8-liter feed tank. The liquor concentration was 44.2 g/100 g and the pH was adjusted to 5.5 with 1 M NaOH. The hydrolysate was composed as set forth in Table 12-1, whereby the HPLC analyses are given on peak area-% basis.

TABLE 12-1

| Composition of Feed | % |
|---|---|
| L-fucose | 22.5 |
| Galactose + Glucose | 8.5 |
| Lactose | 55.5 |
| 2′fucosyllactose | 5.8 |
| Difucosyllactose | 0.0 |
| Others | 7.8 |

The feed was heated to 56° C. and water was used for diafiltration. The filtration pressure was set at 30 Bar and concentrate DS concentration controlled to keep flux at greater than 6 kg/m²/h.

After batch filtration two permeate fractions and final concentrate fraction were collected. The result including HPLC analyses on peak area-% basis for the permeate fractions, final concentrate and combined evaporated permeate are set forth in Table 12-2.

TABLE 12-2

| | | | Final |
|---|---|---|---|
| | Permeate 1 | Permeate 2 | concentrate |
| mass, kg | 15.2 | 16.5 | 6.9 |
| Dry substance, g/100 g | 2.4 | 0.9 | 16.1 |
| L-fucose, % | 64.0 | 52.1 | 2.8 |
| Galactose + Glucose, % | 22.0 | 22.2 | 1.6 |
| Lactose, % | 13.2 | 24.5 | 75.7 |
| 2′fucosyllactose, % | 0.0 | 0.0 | 8.3 |
| Difucosyllactose, % | 0.0 | 0.0 | 0.0 |
| Others, % | 0.9 | 1.1 | 11.7 |

The overall L-fucose yield calculated from the permeate fractions was 91.2%.

### Example 13

### Nanofiltration of 2′-FL Acid Hydrolysate

The process equipment included a plate and frame filtration unit (Alfa Laval Labstak M20), feed and diafiltration pump, heat exchanger, heating water bath, 8-liter feed tank as well as inlet and outlet pressure gauges and pressure control valve. The total membrane area was 0.72 m². The membrane installed was Desal 5 DK series (Suez) with an approximate molecular weight cut-off of 150-300 Dalton and 98-99% MgSO₄ retention at 25° C.

As a feed, neutralized HMO hydrolysate from 1.5 pH acid hydrolysis of high purity 2′-FL syrup was used, and the aim was to separate L-fucose contained therein to the NF permeate while retaining lactose.

2.26 kg of feed was fed to an 8-liter feed tank. The liquor concentration was 45.2 g/100 g and the pH was adjusted to 7.8 with 1 M NaOH. The hydrolysate was composed as set forth in Table 13-1, whereby the HPLC analyses are given on peak area-% basis.

TABLE 13-1

| Composition of Feed | % |
|---|---|
| L-fucose | 25.6 |
| Galactose + Glucose | 6.2 |
| Lactose | 53.0 |
| 2′fucosyllactose | 10.8 |
| Difucosyllactose | 0.0 |
| Others | 4.4 |

The feed was heated to 55° C. and water was used for diafiltration. The filtration pressure was set at 30 Bar and concentrate DS concentration controlled to keep flux at greater than 6 kg/m²/h.

After batch filtration, a permeate fraction and final concentrate fraction were collected. The result including HPLC analyses on peak area-% basis for the permeate fraction, evaporated permeate and final concentrate are set forth in Table 13-2.

TABLE 13-2

| | | Evaporated | Final |
|---|---|---|---|
| | Permeate | permeate | concentrate |
| mass, kg | 18.2 | 0.7 | 3.8 |
| Dry substance, g/100 g | 1.7 | 44.5 | 19.4 |
| L-fucose, % | 70.7 | 70.7 | 4.6 |
| Galactose + Glucose, % | 16.2 | 16.1 | 1.6 |
| Lactose, % | 11.7 | 11.8 | 70.3 |
| 2′fucosyllactose, % | 1.4 | 1.5 | 15.1 |
| Difucosyllactose, % | 0.0 | 0.0 | 0.0 |
| Others, % | 0.0 | 0.0 | 8.4 |

The overall L-fucose yield calculated from the permeate fraction was 86.9%.

### Example 14

### Nanofiltration of 2′-FL Crystallization Mother Liquor Enzymatic Hydrolysate

The process equipment included a plate and frame filtration unit (Alfa Laval Labstak M20), feed and diafiltration pump, heat exchanger, heating water bath, 8-liter feed tank as well as inlet and outlet pressure gauges and pressure control valve. The total membrane area was 0.72 m². The membrane installed was DK series (Suez) with an approximate molecular weight cut-off of 150-300 Dalton and 98-99% MgSO₄ retention at 25° C.

Hydrolysate prepared according to Example 6 was used as feed for nanofiltration and the aim was to separate L-fucose contained therein to the NF permeate while retaining lactose.

The feed was heated to about 50-60° C. and water was used for diafiltration. The filtration pressure was set at 30 Bar and concentrate DS concentration controlled to keep flux at greater than 6 kg/m²/h.

After batch filtration permeate fraction and final concentrate fraction were collected. The result including HPLC analyses on peak area-% basis for the permeate fractions, final concentrate and combined evaporated permeate are set forth in Table 14-1.

TABLE 14-1

| | Permeate | Final concentrate |
|---|---|---|
| mass, kg | 37.1 | 7.2 |
| Dry substance, g/100 g | 1.5 | 20.0 |
| L-fucose, % | 89.6 | 1.8 |
| Galactose + Glucose, % | 0.0 | 0.0 |
| Lactose, % | 10.2 | 75.2 |
| 2′fucosyllactose, % | 0.0 | 1.6 |
| Difucosyllactose, % | 0.0 | 13.1 |
| Others, % | 0.2 | 8.2 |

L-fucose yield of nanofiltration calculated from the permeate fraction was 95.0%.

### Example 15

### Enzymatic Hydrolysis of Nanofiltration Permeate

Nanofiltration permeate was evaporated to 45 g/100 g concentration. Hydrolysis was carried out for 1.2 kg of feed liquor in a 2-liter vessel with mixing. The pH was adjusted with dilute NaOH to be about 6.0 and 2.7 grams of Enzyme (250 NLU/g_{lactose} of GODO-YNL2 beta-galactosidase, EC 3.2.1.23) was added. Hydrolysis was continued for 4 hr in constant temperature of 40° C. Nanofiltration permeate was composed as set forth in Table 15-1, whereby the HPLC analyses are given on peak area-% basis.

TABLE 15-1

| Composition of hydrolyzation feed | % |
|---|---|
| L-fucose | 89.6 |
| Galactose + Glucose | 0.0 |
| Lactose | 10.2 |
| 2′fucosyllactose | 0.0 |
| Difucosyllactose | 0.0 |
| Others | 0.2 |

Hydrolysate was composed as set forth in Table 15-2, whereby the HPLC analyses are given on peak area-% basis.

TABLE 15-2

| Composition of hydrolysate | % |
|---|---|
| L-fucose | 89.1 |
| Galactose + Glucose | 10.1 |
| Lactose | 0.5 |
| 2′fucosyllactose | 0.0 |
| Difucosyllactose | 0.0 |
| Others | 0.3 |

### Example 16

### Chromatographic Separation of 2′-FL Crystallization Mother Liquor Hydrolysate Using SAC Na⁺ Resin

The process equipment included a separation column, feed and eluent pump, heat exchanger, flow control means for the out-coming liquid as well as inlet and product valves for the process streams. The height of the column was 1.25 m and column had a diameter of 3.1 cm. The column was packed with a strong acid gel type cation exchange resin in Na⁺-form. The divinylbenzene content of the resin was 5.5% and the mean bead size of the resin was 0.35 mm.

As a feed, neutralized HMO hydrolysate was used and the aim was to separate L-fucose contained therein.

The liquor concentration was 27.9 g/100 ml and the pH was 3.5. The hydrolysate was composed as set forth in Table 16-1, whereby the HPLC analyses are given on peak area-% basis.

TABLE 16-1

| Composition of Feed | % |
|---|---|
| L-fucose | 23.3 |
| Galactose + Glucose | 5.4 |
| Lactose | 42.2 |
| 2′fucosyllactose | 17.6 |
| Difucosyllactose | 0.6 |
| Others | 10.9 |

The feed and the eluent were used at a temperature of 60° C. and water was used as the eluent. The feed volume was 70 ml and the flow rate for the feed and elution was 7 ml/min.

After equilibration of the system with three following feeds fractions were drawn from the separation column: residual fraction, two recycle fractions (both sides of the L-fucose peak) and L-fucose product fraction. The result including HPLC analyses on peak area-% basis for the residual fraction, recycle fractions and the L-fucose fraction are set forth in Table 16-2.

TABLE 16-2

| | | Recycle | | Recycle |
|---|---|---|---|---|
| | Residual | 1 | L-fucose | 2 |
| Volume, ml | 285 | 39 | 100 | 15 |
| Dry substance, g/100 ml | 5.0 | 4.4 | 3.1 | 0.4 |
| L-fucose, % | 3.0 | 64.1 | 90.1 | 72.6 |
| Galactose + Glucose, % | 3.0 | 22.7 | 7.7 | 0.8 |
| Lactose, % | 54.0 | 8.8 | 0.4 | 0.1 |
| 2′fucosyllactose, % | 23.8 | 1.7 | 1.1 | 0.4 |
| Difucosyllactose, % | 1.7 | 0.6 | 0.5 | 0.2 |
| Others, % | 14.5 | 2.1 | 0.2 | 25.9 |

The overall L-fucose yield calculated from these fractions was 86.7% with 9.3% recycle ratio.

### Example 17

### Chromatographic Separation of 2′-FL Crystallization Mother Liquor Hydrolysate Using SAC Ca²⁺ Resin

The process equipment included a separation column, feed and eluent pump, heat exchanger, flow control means for the out-coming liquid as well as inlet and product valves for the process streams. The height of the column was 1.25 m and column had a diameter of 3.1 cm. The column was packed with a strong acid gel type cation exchange resin in Ca²⁺-form. The divinylbenzene content of the resin was 5.5% and the mean bead size of the resin was 0.35 mm.

As a feed, neutralized HMO hydrolysate was used and the aim was to separate L-fucose contained therein.

The liquor concentration was 27.7 g/100 ml and the pH was 3.5. The hydrolysate was composed as set forth in Table 17-1, whereby the HPLC analyses are given on peak area-% basis.

TABLE 17-1

| Composition of Feed | % |
|---|---|
| L-fucose | 23.3 |
| Galactose + Glucose | 5.6 |
| Lactose | 42.0 |
| 2′fucosyllactose | 17.8 |
| Difucosyllactose | 1.2 |
| Others | 10.1 |

The feed and the eluent were used at a temperature of 60° C. and water was used as the eluent. The feed volume was 70 ml and the flow rate for the feed and elution was 7 ml/min.

After equilibration of the system with three following feeds fractions were drawn from the separation column: residual fraction, two recycle fractions (both sides of the L-fucose peak) and L-fucose product fraction. The result including HPLC analyses on peak area-% basis for the residual fraction, recycle fractions and the L-fucose fraction are set forth in Table 17-2.

TABLE 17-2

| | | Recycle | | Recycle |
|---|---|---|---|---|
| | Residual | 1 | L-fucose | 2 |
| Volume, ml | 277 | 39 | 139 | 15 |
| Dry substance, g/100 ml | 4.8 | 4.8 | 2.5 | 0.5 |
| L-fucose, % | 3.6 | 50.7 | 78.6 | 39.3 |
| Galactose + Glucose, % | 4.3 | 12.9 | 5.0 | 0.7 |
| Lactose, % | 53.8 | 22.7 | 6.9 | 3.4 |
| 2′fucosyllactose, % | 23.7 | 5.3 | 2.1 | 0.5 |
| Difucosyllactose, % | 1.1 | 0.0 | 0.0 | 0.0 |
| Others, % | 13.5 | 8.4 | 7.4 | 56.1 |

The overall L-fucose yield calculated from these fractions was 85.1% with 10.4% recycle ratio.

### Example 18

### Chromatographic Separation of Nanofiltration Permeate Using SAC Na⁺ Resin

The process equipment included a separation column, feed and eluent pump, heat exchanger, flow control means for the out-coming liquid as well as inlet and product valves for the process streams. The height of the resin bed was 1.57 m and column had a diameter of 9.3 cm. The column was packed with a strong acid gel type cation exchange resin in Nat-form. The divinylbenzene content of the resin was 5.5% and the mean bead size of the resin was 0.35 mm.

As a feed, neutralized HMO hydrolysate, which was nanofiltered, was used and the aim was to separate L-fucose contained therein.

The liquor concentration was 27.7 g/100 ml and the pH was 3.5. The hydrolysate was composed as set forth in Table 18-1, whereby the HPLC analyses are given on peak area-% basis.

TABLE 18-1

| Composition of Feed | % |
|---|---|
| L-fucose | 61.8 |
| Galactose + Glucose | 17.7 |
| Lactose | 16.2 |
| 2′fucosyllactose | 0.9 |
| Difucosyllactose | 0.0 |
| Others | 3.4 |

The feed and the eluent were used at a temperature of 60° C. and water was used as the eluent. The feed volume was 800 ml and the flow rate for the feed and elution was 50 ml/min.

After equilibration of the system with three following feeds fractions were drawn from the separation column: residual fraction, two recycle fractions (both sides of the L-fucose peak) and L-fucose product fraction. The result including HPLC analyses on peak area-% basis for the residual fraction, recycle fractions and the L-fucose fraction are set forth in Table 18-2.

TABLE 18-2

| | | Recycle | | Recycle |
|---|---|---|---|---|
| | Residual | 1 | L-fucose | 2 |
| Volume, ml | 3.4 | 0.35 | 1.05 | 0.08 |
| Dry substance, g/100 ml | 2.4 | 10.1 | 10.4 | 0.6 |
| L-fucose, % | 11.1 | 63.4 | 90.1 | 75.9 |
| Galactose + Glucose, % | 22.3 | 32.5 | 7.7 | 0.4 |
| Lactose, % | 49.7 | 0.7 | 0.5 | 0.2 |
| 2′fucosyllactose, % | 4.5 | 0.6 | 0.4 | 0.2 |
| Difucosyllactose, % | 0.0 | 0.0 | 0.0 | 0.0 |
| Others, % | 12.4 | 2.8 | 1.3 | 23.3 |

The overall L-fucose yield calculated from these fractions was 91.6% with 15.8% recycle ratio.

### Example 19

### Chromatographic Separation of 2′-FL Crystallization Mother Liquor Hydrolysate Using SAC Na⁺ Resin

The process equipment included a separation column, feed and eluent pump, heat exchanger, flow control means for the out-coming liquid as well as inlet and product valves for the process streams. The height of the resin bed was 1.57 m and column had a diameter of 9.3 cm. The column was packed with a strong acid gel type cation exchange resin in Nat-form. The divinylbenzene content of the resin was 5.5% and the mean bead size of the resin was 0.35 mm.

As a feed, neutralized HMO hydrolysate was used and the aim was to separate L-fucose contained therein.

The liquor concentration was 27.7 g/100 ml and the pH was 3.5. The hydrolysate was composed as set forth in Table 19-1, whereby the HPLC analyses are given on peak area-% basis.

TABLE 19-1

| Composition of Feed | % |
|---|---|
| L-fucose | 22.3 |
| Galactose + Glucose | 8.6 |
| Lactose | 55.9 |
| 2′fucosyllactose | 5.8 |
| Difucosyllactose | 0.0 |
| Others | 7.4 |

The feed and the eluent were used at a temperature of 60° C. and water was used as the eluent. The feed volume was 800 ml and the flow rate for the feed and elution was 50 ml/min.

After equilibration of the system with three following feeds fractions were drawn from the separation column: residual fraction, two recycle fractions (both sides of the L-fucose peak) and L-fucose product fraction. The result including HPLC analyses on peak area-% basis for the residual fraction, recycle fractions and the L-fucose fraction are set forth in Table 19-2

TABLE 19-2

| | | Recycle | | Recycle |
|---|---|---|---|---|
| | Residual | 1 | L-fucose | 2 |
| Volume, l | 3.4 | 0.3 | 0.9 | 0.2 |
| Dry substance, g/100 ml | 5.3 | 5.2 | 3.8 | 0.4 |
| L-fucose, % | 2.3 | 65.9 | 91.3 | 48.4 |
| Galactose + Glucose, % | 6.2 | 31.4 | 7.9 | 0.4 |
| Lactose, % | 68.4 | 1.2 | 0.7 | 0.2 |
| 2′fucosyllactose, % | 10.0 | 0.3 | 0.1 | 0.1 |
| Difucosyllactose, % | 0.0 | 0.0 | 0.0 | 0.0 |
| Others, % | 13.1 | 1.2 | 0.0 | 50.9 |

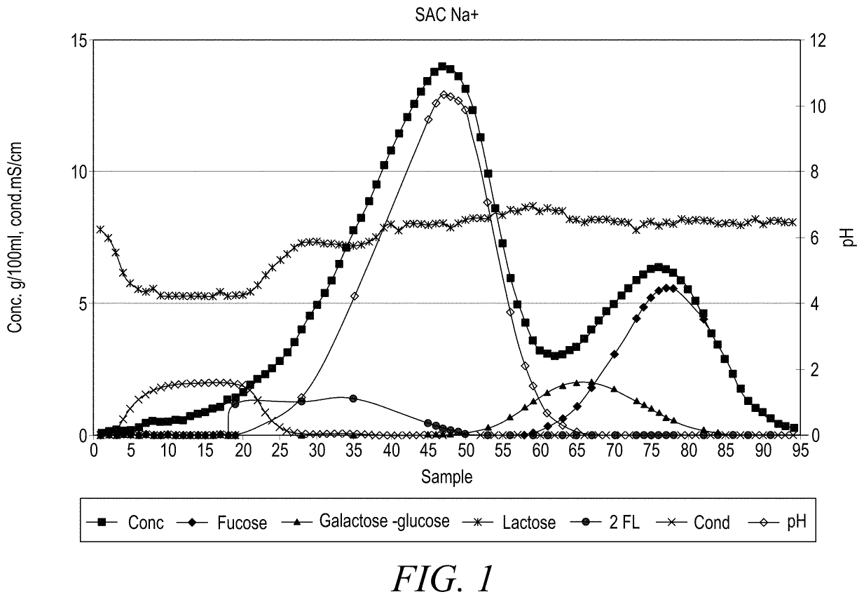

The overall L-fucose yield calculated from these fractions was 88.3% with 7.1% recycle ratio. FIG. 1 is a graphical presentation of the elution profile.

### Example 20

### Chromatographic Separation of Hydrolyzed 2′-FL Crystal Cake Using SAC Na⁺ Resin

The process equipment included a separation column, feed and eluent pump, heat exchanger, flow control means for the out-coming liquid as well as inlet and product valves for the process streams. The height of the resin bed was 1.57 m and column had a diameter of 9.3 cm. The column was packed with a strong acid gel type cation exchange resin in Na⁺-form. The divinylbenzene content of the resin was 5.5% and the mean bead size of the resin was 0.35 mm.

As a feed, neutralized HMO hydrolysate was used and the aim was to separate L-fucose contained therein.

The liquor concentration was 27.6 g/100 ml and the pH was 3.5. The hydrolysate was composed as set forth in Table 20-1, whereby the HPLC analyses are given on peak area-% basis.

TABLE 20-1

| Composition of Feed | % |
|---|---|
| L-fucose | 24.9 |
| Galactose + Glucose | 6.1 |
| Lactose | 52.6 |
| 2′fucosyllactose | 10.7 |
| Difucosyllactose | 0.0 |
| Others | 5.7 |

The feed and the eluent were used at a temperature of 60° C. and water was used as the eluent. The feed volume was 800 ml and the flow rate for the feed and elution was 50 ml/min.

After equilibration of the system with three following feeds fractions were drawn from the separation column: residual fraction, two recycle fractions (both sides of the L-fucose peak) and L-fucose product fraction. The result including HPLC analyses on peak area-% basis for the residual fraction, recycle fractions and the L-fucose fraction are set forth in Table 20-2.

TABLE 20-2

| | | Recycle | | Recycle |
|---|---|---|---|---|
| | Residual | 1 | L-fucose | 2 |
| Volume, l | 3.2 | 0.4 | 1.0 | 0.1 |
| Dry substance, g/100 ml | 5.1 | 3.7 | 4.9 | 0.8 |
| L-fucose, % | 1.4 | 58.8 | 92.0 | 66.5 |
| Galactose + Glucose, % | 3.0 | 38.4 | 7.9 | 0.2 |
| Lactose, % | 70.4 | 2.6 | 0.1 | 0.1 |
| 2′fucosyllactose, % | 17.2 | 0.2 | 0.0 | 0.0 |
| Difucosyllactose, % | 0.0 | 0.0 | 0.0 | 0.0 |
| Others, % | 8.0 | 0.0 | 0.0 | 33.2 |

The overall L-fucose yield calculated from these fractions was 94.9% with 6.2% recycle ratio.

### Example 21

### SMB Separation of 2′-FL Crystallization Mother Liquor Hydrolysate Using SAC Na⁺ Resin

The test equipment included four columns connected in series, feed pump, recycling pumps, eluent water pump as well as inlet and product valves for the various process streams. The height of each column was 4 m and each column had a diameter of 0.111 m. The columns were packed with a strong acid gel type cation exchange resin in Na+-form. The divinylbenzene content of the resin was 5.5% and the mean bead size of the resin was 0.35 mm.

As a feed, neutralized HMO hydrolysate was used and the aim was to separate L-fucose contained therein. The liquor concentration was 45.5 g/100 ml and the pH was 3.3. The hydrolysate was composed as set forth in Table 21-1, whereby the HPLC analyses are given on peak area-% basis.

TABLE 21-1

| Composition of Feed | % |
|---|---|
| L-fucose | 23.1 |
| Galactose + Glucose | 3.4 |
| Lactose | 43.6 |
| 2′fucosyllactose | 5.9 |
| Difucosyllactose | 0.3 |
| Others | 23.7 |

The fractionation was performed by way of a 7-step SMB sequence as set forth below. The feed and the eluent were used at a temperature of 65° C. and water was used as an eluent.

Step 1: 6.0 liters of feed solution was pumped into the first column at a flow rate of 21 l/h and Residual fraction was collected from the fourth column.

Step 2: 7.0 l of feed solution was pumped into the first column at a flow rate of 21 l/h and first 4.0 l of Fucose1 fraction and then 3.0 l of Fucose2 fraction were collected from the fourth column.

Step 3: 6.0 l of water was pumped into the first column at a flow rate of 21 l/h and a Fucose2 fraction was collected from the fourth column.

Step 4: 2.0 l of water was pumped into the first column at a flow rate of 21 l/h and a Recycle fraction was collected from the fourth column.

Step 5: 4.0 l were circulated in the column set loop, formed with all columns, at a flow rate of 21 l/h.

Step 6: 25.0 l of water was pumped into the first column at a flow rate of 21 l/h and a Residual fraction was collected from the second column. Simultaneously 16.5 l of water was pumped into the third column at a flow rate of 13.9 l/h and a Residual fraction was collected from the fourth column.

Step 7: 8.0 l of water was pumped into the first column at a flow rate of 21 l/h and a Residual fraction was collected from the fourth column.

After equilibration of the system, the following fractions were drawn from the system: Residual fractions from columns two and four, L-fucose containing fractions and recycle fraction from the fourth column. The results including HPLC analyses for combined residual fraction and other collected fractions are set forth in Table 21-2.

TABLE 21-2

| | | | L-fucose | L-fucose |
|---|---|---|---|---|
| | Residual | Recycle | 1 | 2 |
| Volume, l | 55.5 | 2.0 | 4.0 | 9.0 |
| Dry substance, g/100 ml | 8.6 | 0.2 | 14.1 | 7.7 |
| L-fucose, % | 5.5 | 91.2 | 84.9 | 96.4 |
| Galactose + Glucose, % | 3.0 | 0.0 | 11.6 | 2.2 |
| Lactose, % | 56.4 | 6.0 | 0.1 | 0.1 |
| 2′fucosyllactose, % | 7.1 | 0.0 | 0.0 | 0.0 |
| Difucosyllactose, % | 0.3 | 0.0 | 0.0 | 0.0 |
| Others, % | 27.7 | 2.8 | 3.4 | 1.3 |

The overall L-fucose yield calculated from these fractions was 81.3% and purity for the combined L-fucose fraction was 91.3%.

### Example 22

### Chromatographic Separation of 2′-FL Crystallization Mother Liquor Hydrolysate Using WAC H⁺ Resin

The process equipment included a separation column, feed and eluent pump, heat exchanger, flow control means for the out-coming liquid as well as inlet and product valves for the process streams. The height of the resin bed was 1.57 m and column had a diameter of 9.3 cm. The column was packed with a weak acid gel type cation exchange resin in H-form. The divinylbenzene content of the resin was 8.0% and the mean bead size of the resin was 0.39 mm.

As a feed, neutralized HMO hydrolysate was used and the aim was to separate L-fucose contained therein.

The liquor concentration was 26.1 g/100 ml and the pH was 3.5. The hydrolysate was composed as set forth in Table 22-1, whereby the HPLC analyses are given on peak area-% basis.

TABLE 22-1

| Composition of Feed | % |
|---|---|
| L-fucose | 24.0 |
| Galactose + Glucose | 3.4 |
| Lactose | 43.0 |
| 2′fucosyllactose | 5.0 |
| Difucosyllactose | 0.2 |
| Others | 24.4 |

The feed and the eluent were used at a temperature of 60° C. and water was used as the eluent. The feed volume was 800 ml and the flow rate for the feed and elution was 50 ml/min.

After equilibration of the system with five following feeds fractions were drawn from the separation column: residual fraction, recycle fraction (before the L-fucose peak) and L-fucose product fraction. The result including HPLC analyses on peak area-% basis for the residual fraction, recycle fraction and the L-fucose fraction are set forth in Table 22-2.

TABLE 22-2

| | Residual | Recycle | L-fucose |
|---|---|---|---|
| Volume, l | 3.3 | 0.3 | 1.5 |
| Dry substance, g/100 ml | 4.5 | 4.3 | 2.9 |
| L-fucose, % | 1.6 | 47.1 | 91.1 |
| Galactose + Glucose, % | 3.2 | 11.3 | 1.7 |
| Lactose, % | 60.0 | 11.4 | 0.8 |
| 2′fucosyllactose, % | 8.2 | 1.0 | 0.4 |
| Difucosyllactose, % | 0.7 | 0.5 | 0.4 |
| Others, % | 26.3 | 28.7 | 5.6 |

The overall L-fucose yield calculated from these fractions was 94.4% with 6.3% recycle ratio.

### Example 23

### Chromatographic Separation of Hydrolyzed 3-FL Syrup Using SAC Na⁺ Resin

The process equipment included a separation column, feed and eluent pump, heat exchanger, flow control means for the out-coming liquid as well as inlet and product valves for the process streams. The height of the resin bed was 1.57 m and column had a diameter of 9.3 cm. The column was packed with a weak acid gel type cation exchange resin in H⁺-form. The divinylbenzene content of the resin was 5.5% and the mean bead size of the resin was 0.35 mm.

As a feed, neutralized HMO hydrolysate was used and the aim was to separate L-fucose contained therein.

The liquor concentration was 26.3 g/100 ml and the pH was 3.2. The hydrolysate was composed as set forth in Table 23-1, whereby the HPLC analyses are given on peak area-% basis.

TABLE 23-1

| Composition of Feed | % |
|---|---|
| L-fucose | 24.4 |
| Galactose + Glucose | 5.3 |
| Lactose | 52.7 |
| 3-fucosyllactose | 6.7 |
| Difucosyllactose | 0.0 |
| Others | 10.9 |

The feed and the eluent were used at a temperature of 60° C. and water was used as the eluent. The feed volume was 800 ml and the flow rate for the feed and elution was 50 ml/min.

After equilibration of the system with five following feeds fractions were drawn from the separation column: residual fraction, recycle fraction (before the L-fucose peak) and L-fucose product fraction. The result including HPLC analyses on peak area-% basis for the residual fraction, recycle fraction and the L-fucose fraction are set forth in Table 23-2.

TABLE 23-2

| | Residual | Recycle | L-fucose |
|---|---|---|---|
| Volume, l | 3.4 | 0.5 | 1.1 |
| Dry substance, g/100 ml | 4.5 | 3.6 | 3.8 |
| L-fucose, % | 1.9 | 61.4 | 90.8 |
| Galactose + Glucose, % | 0.6 | 26.1 | 8.0 |
| Lactose, % | 70.1 | 3.5 | 1.0 |
| 3-fucosyllactose, % | 10.8 | 0.3 | 0.2 |
| Difucosyllactose, % | 0.0 | 0.0 | 0.0 |
| Others, % | 16.6 | 8.7 | 0.0 |

The overall L-fucose yield calculated from these fractions was 92.9% with 8.5% recycle ratio.

### Example 24

### Crystallization of L-Fucose from Chromatographically Enriched Syrup at a Temperature of 40-69° C.

The crystallization feed material was chromatographically enriched fucose syrup prepared in accordance with Example 19. The RDS, the pH, the color, and the sugar composition of the syrup are given in Table 24-1.

The feed syrup was evaporated to a RDS of 83.3% (Rotavapor R-151 evaporator). The resulting syrup (0.30 kg) was moved to a 1 liter cooling crystallizer, and seeded with 0.035 g of fucose seed crystals at a temperature of 69° C. (seed crystals prepared in accordance with EP 1664352, supersaturation of 1.17). The seeded syrup was cooled to 40° C. in 66 hr under stirring, and then kept stirring at constant temperature for 3 hr.

After 69 hr from seeding, the crystal mass (198 g) was centrifuged with a batch-wise centrifuge having 22.5 cm basket diameter. The amount of wash water was 6 g, the rotating speed was 2150 rpm, and the centrifugation time was 7 min. The centrifugation L-fucose yield was 69.8%.

The centrifuge feed mass RDS was 86.3% due to slow evaporation during cooling. The mother liquor RDS was 74.4% after 67 hr from seeding at 40° C. This correspond to crystal contents of 54% on DS.

A sample of the wet centrifugation cake (70 g) was dried in a heating chamber at 41° C. for 5 hr. The moisture content of the non-dried centrifugation cake was 1.4%. A sample of the non-dried centrifugation cake (21 g) was washed with ethanol and dried. The compositions of the dried crystal sample, the dried ethanol-washed crystal sample, and the centrifugation run-off are given in Table 24-1.

TABLE 24-1

| | | | | | Crys- |
|---|---|---|---|---|---|
| | | | Cen- | | tals |
| | | | trifu- | Dried | washed |
| | | Feed | gation | crys- | with |
| analysis | Process | syrup | run-off | tals | ethanol |
| RDS, % | refractometer | 64.0 | 80.9 | n/a | n/a |
| moisture, % | coulometric | n/a | n/a | 0.1 | 0.1 |
| pH, — (10 | Karl Fischer | 4.3 | 4.1 | 5.1 | n/a |
| wt % solution) | pH meter | | | | |
| color, ICUMSA | ICUMSA | 148 | 382 | 22 | n/a |
| | Spectro- | | | | |
| | photometric | | | | |
| L-Fucose, | HPLC, | 90.1 | 78.7 | 99.0 | 100.0 |
| area-% | NH₂-column | | | | |
| L-Fucose, % | HPLC, | 89.8 | 78.8 | 99.0 | 99.9 |
| on RDS | NH₂-column | | | | |
| Galactose, % | HPLC, | 6.9 | 14.8 | 0.4 | 0.0 |
| on RDS | NH₂-column | | | | |
| Glucose, % | HPLC, | 1.2 | 2.2 | 0.0 | 0.0 |
| on RDS | NH₂-column | | | | |
| Others, % | HPLC, | 0.9 | 2.7 | 0.3 | 0.0 |
| on RDS | NH₂-column | | | | |

The melting point (m.p.) was measured by using European Pharmacopoeia process same way as in the EP 1664352. The melting point of the crystals was 139.0-141.0° C. and ethanol washed crystals 139.8-141.7° C. Specific optical rotation of the dried crystals was −74.5° (water, c=2, 20° C.). The optical rotation was measured from 2 g/100 ml water solution at 20° C. by using Anton Paar MCP 300 Sucromet, cuvette 100 mm, Na 589 light.

### Example 25

### Crystallization of L-Fucose from Chromatographically Enriched Syrup at a Temperature of 44-75° C.

The crystallization feed material was chromatographically enriched fucose syrup prepared in accordance with Example 21. The RDS, the pH, the color, and the sugar composition of the syrup are given in Table 25-1.

TABLE 25-1

| analysis | Process | Feed syrup |
|---|---|---|
| RDS, % | refractometer | 59.1 |
| pH, — (10 | pH meter | 5.6 |
| wt % solution) | | |
| color, ICUMSA | ICUMSA | 16 |
| | Spectrophotometric | |
| L-Fucose, area-% | HPLC, HILIC | 91.2 |
| L-Fucose, % on RDS | HPLC, HILIC | 90.4 |
| Galactose and | HPLC, HILIC | 6.5 |
| glucose, % on RDS | | |
| Lactose, % on RDS | HPLC, HILIC | 0.0 |
| Others, % on RDS | HPLC, HILIC | 2.2 |
| L-Fucose, area-% | HPLC, NH₂-column | 90.9 |
| L-Fucose, % on RDS | HPLC, NH₂-column | 90.1 |
| Galactose, % on RDS | HPLC, NH₂-column | 5.8 |
| Glucose, % on RDS | HPLC, NH₂-column | 0.9 |
| Others, % on RDS | HPLC, NH₂-column | 2.2 |

The feed syrup was evaporated to a RDS of 87.1% (Rotavapor R-151 evaporator). The resulting syrup (12.4 kg) was moved to a 9 liters cooling crystallizer, and seeded with 1.2 g of fucose seed crystals at a temperature of 75° C. (seed crystals prepared in accordance with Example 24, supersaturation of 1.41). The seeded syrup was cooled to 44° C. within 38 hr under stirring, and then kept stirring at constant temperature for 4 hr. After 22 hr and 39 hr from seeding, small samples of the crystal mass were centrifuged without washing (Hettich Rotanta 460R centrifuge, 1640 rpm, 10 min) to monitor progression of crystallization. The mother liquor DS was 80.4% after 22 hr from seeding and 75.7% after 39 hr from seeding. These values correspond to crystal contents of 39% on DS and 54% on DS, respectively. A microscopic image of the crystal mass after 38 hr from seeding representing large crystal size and cubic crystal habit is shown in FIG. 2.

The crystal mass (10.4 kg) was centrifuged with a batch-wise centrifuge having 40.5 cm basket diameter. The amount of wash water was 350 g, the rotating speed was 1600 rpm, and the centrifugation time was 7 min. The centrifugation L-fucose yield was 59%.

A sample of the wet centrifugation cake (27 g) was dried in a heating chamber at 40° C. for 21 hr. The moisture content of the non-dried centrifugation cake was 2.3%. A sample of the non-dried centrifugation cake (25 g) was washed with ethanol and dried. The compositions of the dried crystal sample, the dried ethanol-washed crystal sample, and the centrifugation run-off are given in Table 25-2.

TABLE 25-2

| | | | | Crys- |
|---|---|---|---|---|
| | | Cen- | | tals |
| | | trifu- | Dried | washed |
| | | gation | crys- | with |
| analysis | Process | run-off | tals | ethanol |
| RDS , % | refractometer | 73.5 | 100.0 | 99.7 |
| moisture, % | coulometric | n/a | 0.2 | n/a |
| | Karl Fischer | | | |
| pH, — (10 | pH meter | 4.3 | 5.5 | n/a |
| wt % solution) | | | | |
| color, ICUMSA | ICUMSA | 239 | 8 | n/a |
| | Spectro- | | | |
| | photometric | | | |
| L-Fucose, area-% | HPLC, HILIC | 80.6 | 99.7 | 100.0 |
| L-Fucose, % on RDS | HPLC, HILIC | 80.6 | 99.7 | 101.4 |
| Galactose and | HPLC, HILIC | 14.1 | 0.1 | 0.0 |
| glucose, % on RDS | | | | |
| Lactose, % on RDS | HPLC, HILIC | 0.2 | 0.0 | 0.0 |
| Others, % on RDS | HPLC, HILIC | 4.3 | 0.2 | 0.0 |
| L-Fucose, area-% | HPLC, NH₂-column | 80.7 | 99.9 | 99.9 |
| L-Fucose, % on RDS | HPLC, NH₂-column | 79.3 | 98.9 | 99.8 |
| Galactose, % on RDS | HPLC, NH₂-column | 12.6 | 0.0 | 0.0 |
| Glucose, % on RDS | HPLC, NH₂-column | 1.4 | 0.0 | 0.0 |
| Others, % on RDS | HPLC, NH₂-column | 3.9 | 0.1 | 0.2 |

The melting point (m.p.) was measured by using European Pharmacopoeia process same way as in the EP 1664352. The melting point of the crystals was 139.3-140.0° C.

### Example 26

### Crystallization of L-Fucose from Chromatographically Enriched Syrup at a Temperature of 25-55° C.

The crystallization feed material was chromatographically enriched fucose syrup prepared in accordance with Example 21. The RDS, the pH, the color, and the sugar composition of the syrup are given in Table 25-1.

The feed syrup was evaporated to a RDS of 80.8% (Rotavapor R-151 evaporator). The syrup (12.6 kg) was seeded with 1.7 g of fucose seed crystals at a temperature of 55° C. (seed crystals prepared in accordance with patent EP1664352, supersaturation of 1.30). The seeded syrup was boiled at a temperature of 51-54° C. and at pressure of 70 mbar for 40 min. The RDS of the resulting crystal mass was 83.0%.

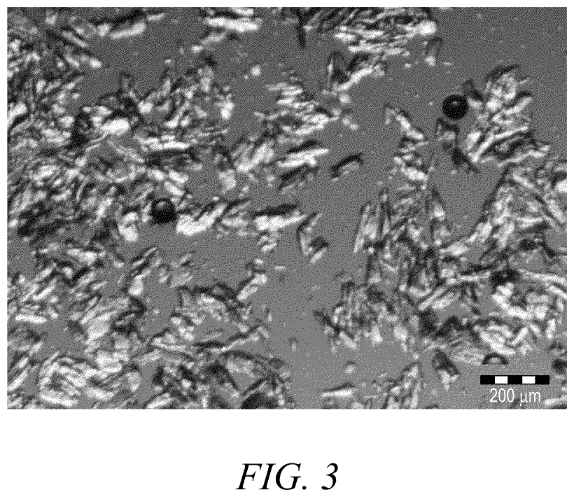

The crystal mass was moved to a 9 liters cooling crystallizer, and cooled to 25° C. within 40 hr under stirring. After cooling, the crystal mass was kept stirring at constant temperature for 5 hr. The mother liquor DS was 73.0% after 20 hr from seeding and 69.1% after 41 hr from seeding, which correspond to crystal contents of 45% on DS and 54% on DS, respectively. A microscopic image of the crystal mass after 41 hr from seeding representing small crystal size and needlelike crystal habit is shown in FIG. 3.

The crystal mass was divided into three parts. The first part (1.15 kg) was centrifuged with a batch-wise centrifuge having 22.5 cm basket diameter, the second part (7.78 kg) was centrifuged with a batch-wise centrifuge having 40.5 cm basket diameter, and the third part (1.25 kg) was moved to a 2 liters crystallizer to continue crystallization with a mixture of water and ethanol as solvent (Example 27).

In the first centrifugation experiment (basket diameter 22.5 cm, wash water 50 mL, 3350 rpm, 7 min), the centrifugation L-fucose yield was 59%, and the moisture content of the non-dried centrifugation cake was 5.1%. The compositions of the dried crystal sample, the ethanol-washed crystal sample, and the centrifugation run-off are given in Table 26-1.

TABLE 26-1

| | | | | Crys- |
|---|---|---|---|---|
| | | Cen- | | tals |
| | | trifu- | Dried | washed |
| | | gation | crys- | with |
| analysis | Process | run-off | tals | ethanol |
| RDS, % | refractometer | 67.9 | 100.0 | 99.9 |
| moisture, % | coulometric | n/a | 0.3 | n/a |
| | Karl Fischer | | | |
| pH, — (10 | pH meter | 4.7 | 5.5 | n/a |
| wt % solution) | | | | |
| color, ICUMSA | ICUMSA | 70 | 7 | n/a |
| | Spectro- | | | |
| | photometric | | | |
| L-fucose, area-% | HPLC, HILIC | 81.5 | 99.0 | 100.0 |
| L-Fucose, % on RDS | HPLC, HILIC | 80.7 | 99.1 | 100.6 |
| Galactose and | HPLC, HILIC | 13.8 | 1.2 | 0.0 |
| glucose, % on RDS | | | | |
| Lactose, % on RDS | HPLC, HILIC | 0.5 | 0.0 | 0.0 |
| Others, % on RDS | HPLC, HILIC | 5.7 | 1.2 | 0.0 |
| L-Fucose, area-% | HPLC, NH₂-column | 81.7 | 98.8 | 99.8 |
| L-Fucose, % on RDS | HPLC, NH₂-column | 79.9 | 97.7 | 98.7 |
| Galactose, % on RDS | HPLC, NH₂-column | 11.4 | 0.8 | 0.0 |
| Glucose, % on RDS | HPLC, NH₂-column | 1.4 | 0.2 | 0.0 |
| Others, % on RDS | HPLC, NH₂-column | 3.7 | 0.6 | 0.1 |

The melting point (m.p.) was measured by using European Pharmacopoeia process same way as in the EP 1664352. The melting point of the crystals was 140.3-141.5° C.

In the second centrifugation (basket diameter 40.5 cm, wash water 260 mL, 1600 rpm, 7 min), the centrifugation L-fucose yield was 56%, and the moisture content of the non-dried centrifugation cake was 6.9%. The compositions of the dried crystal sample, the dried ethanol-washed crystal sample, and the centrifugation run-off are given in Table 26-2.

TABLE 26-2

| | | | | Crys- |
|---|---|---|---|---|
| | | Cen- | | tals |
| | | trifu- | Dried | washed |
| | | gation | crys- | with |
| analysis | Process | run-off | tals | ethanol |
| RDS, % | refractometer | 66.9 | 99.8 | 99.9 |
| moisture, % | coulometric | n/a | 0.4 | n/a |
| | Karl Fischer | | | |
| pH, — (10 | pH meter | 4.8 | 5.2 | n/a |
| wt % solution) | | | | |
| color, ICUMSA | ICUMSA | 67 | 8 | n/a |
| | Spectro- | | | |
| | photometric | | | |
| L-Fucose, area-% | HPLC, HILIC | 82.5 | 98.7 | 100.0 |
| L-Fucose, % on RDS | HPLC, HILIC | 81.5 | 98.9 | 100.1 |
| Galactose and | HPLC, HILIC | 13.1 | 1.4 | 0.0 |
| glucose, % on RDS | | | | |
| Lactose, % on RDS | HPLC, HILIC | 0.5 | 0.0 | 0.0 |
| Others, % on RDS | HPLC, HILIC | 5.5 | 1.2 | 0.0 |
| L-Fucose, area-% | HPLC, NH₂-column | 81.8 | 98.9 | 100.0 |
| L-Fucose, % on RDS | HPLC, NH₂-column | 80.0 | 97.5 | 98.8 |
| Galactose, % on RDS | HPLC, NH₂-column | 10.7 | 0.9 | 0.0 |
| Glucose, % on RDS | HPLC, NH₂-column | 1.2 | 0.1 | 0.0 |
| Others, % on RDS | HPLC, NH₂-column | 5.6 | 0.9 | 0.0 |

The melting point (m.p.) was measured by using European Pharmacopoeia process same way as in the EP 1664352. The melting point of the crystals was 139.6-140.4° C.

### Example 27

### Crystallization of L-Fucose from Chromatographically Enriched Syrup with a Mixture of Water and Ethanol as Solvent

The crystal mass sample (1.25 kg) from Example 26 was kept stirring in a 2 liters crystallizer at 25° C., and 92 g of ethanol was mixed into the mass to reduce the viscosity. The resulting mass was cooled to 16° C. within 12 hr under stirring, and kept stirring at constant temperature for 14 hr.

The crystal mass (1.13 kg) was centrifuged with 42 mL wash water (batch-wise centrifuge, basket diameter 22.5 cm, 2830 rpm, 7 min). The centrifugation L-fucose yield was 60%.

The compositions of the dried crystal sample, the dried ethanol-washed crystal sample, and the centrifugation run-off are given in Table 27-1.

TABLE 27-1

| | | | | Crys- |
|---|---|---|---|---|
| | | Cen- | | tals |
| | | trifu- | Dried | washed |
| | | gation | crys- | with |
| analysis | Process | run-off | tals | ethanol |
| RDS, % | refractometer | n/a | 99.9 | 100.0 |
| moisture, % | coulometric | n/a | 0.3 | n/a |
| | Karl Fischer | | | |
| pH, — (10 | pH meter | 5.0 | 5.5 | n/a |
| wt % solution) | | | | |
| color, ICUMSA | ICUMSA | 71 | 6 | n/a |
| | Spectro- | | | |
| | photometric | | | |
| L-Fucose, area-% | HPLC, HILIC | 81.2 | 99.3 | 100.0 |
| L-Fucose, % on RDS | HPLC, HILIC | 80.3 | 98.1 | 100.2 |
| Galactose and | HPLC, HILIC | 14.0 | 0.9 | 0.0 |
| glucose, % on RDS | | | | |
| Lactose, % on RDS | HPLC, HILIC | 0.3 | 0.0 | 0.0 |
| Others, % on RDS | HPLC, HILIC | 6.0 | 0.5 | 0.0 |
| L-Fucose, area-% | HPLC, NH₂-column | 80.2 | 99.2 | 100.0 |
| L-Fucose, % on RDS | HPLC, NH₂-column | 79.1 | 98.6 | 98.5 |
| Galactose, % on RDS | HPLC, NH₂-column | 11.9 | 0.4 | 0.0 |
| Glucose, % on RDS | HPLC, NH₂-column | 1.5 | 0.0 | 0.0 |
| Others, % on RDS | HPLC, NH₂-column | 4.7 | 0.5 | 0.0 |

The melting point (m.p.) was measured by using European Pharmacopoeia process same way as in the EP 1664352. The melting point of the dried crystals was 140.2-141.0° C.

### Example 28

### Crystallization of L-Fucose from Centrifugation Mother Liquors and Equipment Wash from Examples 25 and 26

The feed material for the crystallization was obtained by combining the centrifugation mother liquors and diluted crystal masses recovered from washing the equipment from Examples 25 and 26. The RDS, the pH, the color, and the sugar composition of the syrup are given in Table 28-1.

TABLE 28-1

| analysis | Process | Crystallization feed |
|---|---|---|
| RDS, % | refractometer | 60.3 |
| pH, — (10 | pH meter | 4.6 |
| wt % solution) | | |
| color, ICUMSA | ICUMSA | 161 |
| | Spectrophotometric | |
| L-Fucose, area-% | HPLC, HILIC | 83.4 |
| L-Fucose, % on RDS | HPLC, HILIC | 83.1 |
| Galactose and | HPLC, HILIC | 12.2 |
| glucose, % on RDS | | |
| Lactose, % on RDS | HPLC, HILIC | 0.7 |
| Others, % on RDS | HPLC, HILIC | 5.5 |
| L-Fucose, area-% | HPLC, NH₂-column | 84.0 |
| L-Fucose, % on RDS | HPLC, NH₂-column | 83.2 |
| Galactose, % on RDS | HPLC, NH₂-column | 10.6 |
| Glucose, % on RDS | HPLC, NH₂-column | 1.3 |
| Others, % on RDS | HPLC, NH₂-column | 3.8 |

The feed syrup was evaporated to a RDS of 87.8% (Rotavapor R-151 evaporator). The resulting syrup (8.4 kg) was moved to a 6 liters cooling crystallizer, and seeded with 2.0 g of fucose seed crystals at a temperature of 75° C. (seed crystals prepared in accordance with Example 24, supersaturation of 1.37). The seeded syrup was cooled to 40° C. within 42 hr under stirring, and then kept stirring at constant temperature for 2 hr. The mother liquor DS was 82.4% after 24 hr from seeding and 78.7% after 40 hr from seeding, which correspond to crystal contents of 35% on DS and 49% on DS, respectively.

The crystal mass (7.1 kg in total) was centrifuged in five batches with wash water at an amount equaling 70-75 mL/kg mass DS (batch-wise centrifuge, basket diameter 22.5 cm, 2690 rpm, 7 min). The average centrifugation L-fucose yield was 56%, and the moisture content of the combined, non-dried cake was 2.9%. The compositions of the dried crystal sample (40° C., 23 hr), the dried ethanol-washed crystal sample, and the centrifugation run-off are given in Table 28-2.

TABLE 28-2

| | | | | Crys- |
|---|---|---|---|---|
| | | Cen- | | tals |
| | | trifu- | Dried | washed |
| | | gation | crys- | with |
| analysis | Process | run-off | tals | ethanol |
| RDS, % | refractometer | 73.4 | 99.9 | 100.0 |
| moisture, % | coulometric | n/a | 0.3 | n/a |
| | Karl Fischer | | | |
| pH, — (10 | pH meter | 4.4 | 5.5 | n/a |
| wt % solution) | | | | |
| color, ICUMSA | ICUMSA | 572 | 23 | n/a |
| | Spectro- | | | |
| | photometric | | | |
| L-Fucose, area-% | HPLC, HILIC | 69.5 | 99.2 | 100.0 |
| L-Fucose, % on RDS | HPLC, HILIC | 68.4 | 99.3 | 100.5 |
| Galactose and | HPLC, HILIC | 21.7 | 0.9 | 0.0 |
| glucose, % on RDS | | | | |
| Lactose, % on RDS | HPLC, HILIC | 0.6 | 0.0 | 0.0 |
| Others, % on RDS | HPLC, HILIC | 8.1 | 0.4 | 0.0 |
| L-Fucose, area-% | HPLC, NH₂-column | 70.1 | 99.7 | 100.0 |
| L-Fucose, % on RDS | HPLC, NH₂-column | 68.5 | 98.9 | 99.8 |
| Galactose, % on RDS | HPLC, NH₂-column | 19.4 | 0.0 | 0.0 |
| Glucose, % on RDS | HPLC, NH₂-column | 2.3 | 0.0 | 0.0 |
| Others, % on RDS | HPLC, NH₂-column | 6.0 | 0.4 | 0.0 |

The melting point (m.p.) was measured by using European Pharmacopoeia process same way as in the EP 1664352. The melting point of the dried crystals was 140.6-142.1° C.

### Example 29

### Recrystallization of L-Fucose from Centrifugation Crystal Cakes of Examples 25, 26, and 28

L-fucose crystals (10.6 kg in total) from Examples 25, 26 and 28 were combined and dissolved in deionized water. The resulting solution was filtrated though a 0.2 μm sterile filter and evaporated to a RDS of 85.1% (Rotavapor R-153 evaporator). The composition of the syrup is given in Table 29-1.

TABLE 29-1

| analysis | Process | Crystallization feed |
|---|---|---|
| RDS, % | refractometer | 85.1 |
| pH, — (10 | pH meter | 5.1 |
| wt % solution) | | |
| color, ICUMSA | ICUMSA | 7 |
| | Spectrophotometric | |
| L-Fucose, area-% | HPLC, HILIC | 99.2 |
| L-Fucose, % on RDS | HPLC, HILIC | 99.7 |
| Galactose and | HPLC, HILIC | 0.9 |
| glucose, % on RDS | | |
| Lactose, % on RDS | HPLC, HILIC | 0.0 |
| Others, % on RDS | HPLC, HILIC | 0.8 |
| L-Fucose, area-% | HPLC, NH₂-column | 99.8 |
| L-Fucose, % on RDS | HPLC, NH₂-column | 98.7 |
| Galactose, % on RDS | HPLC, NH₂-column | 0.0 |
| Glucose, % on RDS | HPLC, NH₂-column | 0.0 |
| Others, % on RDS | HPLC, NH₂-column | 0.2 |

The syrup (12.3 kg) was moved to a 9 liters cooling crystallizer, and seeded with 1.3 g of fucose seed crystals at a temperature of 75° C. (seed crystals prepared in accordance with Example 24, supersaturation of 1.31). The seeded syrup was cooled to 42° C. within 42 hr under stirring, and then kept stirring at constant temperature for 4 hr. The mother liquor DS was 77.3% after 24 hr from seeding and 70.6% after 43 hr from seeding. These correspond to crystal contents of 40% on DS and 58% on DS, respectively.

The crystal mass (10.6 kg) was centrifuged with 350 mL wash water (batch-wise centrifuge, basket diameter 40.5 cm, 1600 rpm, 7 min). The centrifugation L-fucose yield was 57%, and the moisture content of the non-dried cake was 2.8%. The compositions of the dried crystal sample (40° C., 41 hr), the dried ethanol-washed crystal sample, and the centrifugation run-off are given in Table 29-2.

TABLE 29-2

| | | | | Crys- |
|---|---|---|---|---|
| | | Cen- | | tals |
| | | trifu- | Dried | washed |
| | | gation | crys- | with |
| analysis | Process | run-off | tals | ethanol |
| RDS, % | refractometer | 68.1 | 100.1 | 100.0 |
| moisture, % | coulometric | n/a | 0.1 | n/a |
| | Karl Fischer | | | |
| pH, — (10 | pH meter | 4.3 | 5.7 | n/a |
| wt % solution) | | | | |
| color, ICUMSA | ICUMSA | 25 | 2 | n/a |
| | Spectro- | | | |
| | photometric | | | |
| L-Fucose, area-% | HPLC, HILIC | 98.1 | 100.0 | 100.0 |
| L-Fucose, % on RDS | HPLC, HILIC | 98.6 | 99.8 | 100.2 |
| Galactose and | HPLC, HILIC | 1.5 | 0.0 | 0.0 |
| glucose, % on RDS | | | | |
| Lactose, % on RDS | HPLC, HILIC | 0.0 | 0.0 | 0.0 |
| Others, % on RDS | HPLC, HILIC | 0.9 | 0.0 | 0.0 |
| L-Fucose, area-% | HPLC, NH₂-column | 99.3 | 100.0 | 100.0 |
| L-Fucose, % on RDS | HPLC, NH₂-column | 96.8 | 98.8 | 99.3 |
| Galactose, % on RDS | HPLC, NH₂-column | 0.3 | 0.0 | 0.0 |
| Glucose, % on RDS | HPLC, NH₂-column | 0.0 | 0.0 | 0.0 |
| Others, % on RDS | HPLC, NH₂-column | 0.3 | 0.0 | 0.0 |

The melting point (m.p.) was measured by using European Pharmacopoeia process same way as in the EP 1664352. The melting point of the crystals was 140.0-140.1° C.

### Example 30

### Crystallization of L-Fucose from Nanofiltration Permeate Treated with Beta-Galactosidase at a Temperature of 44-75° C.

The crystallization feed material was nanofiltration permeate treated with beta-galactosidase prepared in accordance with Examples 14 and 15. The RDS, the pH, the color, and the sugar composition of the syrup are given in Table 30-1.

TABLE 30-1

| analysis | Process | Feed syrup |
|---|---|---|
| RDS, % | refractometer | 45.1 |
| pH, — (10 | pH meter | 5.5 |
| wt % solution) | | |
| color, ICUMSA | ICUMSA | 21 |
| | Spectrophotometric | |
| L-fucose, area-% | HPLC, HILIC | 89.1 |
| L-Fucose, % on RDS | HPLC, HILIC | 88.7 |
| Galactose and | HPLC, HILIC | 9.9 |
| glucose, % on RDS | | |
| Lactose, % on RDS | HPLC, HILIC | 0.4 |
| Others, % on RDS | HPLC, HILIC | 0.3 |
| L-fucose, area-% | HPLC, NH₂-column | 88.7 |
| L-Fucose, % on RDS | HPLC, NH₂-column | 88.5 |
| Galactose, % on RDS | HPLC, NH₂-column | 5.0 |
| Glucose, % on RDS | HPLC, NH₂-column | 5.0 |
| Others, % on RDS | HPLC, NH₂-column | 0.6 |

The feed syrup was evaporated to a RDS of 87.3% (Rotavapor R-151 evaporator). The resulting syrup (0.56 kg) was moved to a 1-liter cooling crystallizer and seeded with 0.06 g of fucose seed crystals at a temperature of 75° C. (seed crystals prepared in accordance with patent EP1664352, supersaturation of 1.39). The seeded syrup was cooled to 44° C. within 40 hr under stirring, and then kept stirring at constant temperature for 4 hr. The mother liquor DS was 80.7% after 24 hr from seeding and 76.4% after 40 hr from seeding, which correspond to crystal contents of 39% on DS and 53% on DS, respectively.

The crystal mass (0.48 kg) was centrifuged with 16 mL wash water (batch-wise centrifuge, basket diameter 22.5 cm, 2150 rpm, 7 min). The centrifugation L-fucose yield was 58%, and the moisture content of the combined, non-dried cake was 2.4%. The compositions of the dried crystal sample (40° C., 24 hr), the dried ethanol-washed crystal sample, and the centrifugation run-off are given in Table 30-2.

TABLE 30-2

| | | | | Crys- |
|---|---|---|---|---|
| | | Cen- | | tals |
| | | trifu- | Dried | washed |
| | | gation | crys- | with |
| analysis | Process | run-off | tals | ethanol |
| RDS, % | refractometer | 73.7 | 99.9 | 99.8 |
| moisture, % | coulometric | n/a | 0.3 | n/a |
| | Karl Fischer | | | |
| pH, — (10 | pH meter | 4.5 | 5.4 | n/a |
| wt % solution) | | | | |
| color, ICUMSA | ICUMSA | 284 | 10 | n/a |
| | Spectro- | | | |
| | photometric | | | |
| L-Fucose, area-% | HPLC, HILIC | 77.9 | 99.8 | 100.0 |
| L-Fucose, % on RDS | HPLC, HILIC | 77.5 | 99.8 | 100.2 |
| Galactose and | HPLC, HILIC | 20.6 | 0.1 | 0.0 |
| glucose, % on RDS | | | | |
| Lactose, % on RDS | HPLC, HILIC | 0.8 | 0.0 | 0.0 |
| Others, % on RDS | HPLC, HILIC | 0.6 | 0.0 | 0.0 |
| L-Fucose, area-% | HPLC, NH₂-column | 78.0 | 99.9 | 99.9 |
| L-Fucose, % on RDS | HPLC, NH₂-column | 76.8 | 99.1 | 99.6 |
| Galactose, % on RDS | HPLC, NH₂-column | 10.4 | 0.0 | 0.0 |
| Glucose, % on RDS | HPLC, NH₂-column | 10.2 | 0.0 | 0.0 |
| Others, % on RDS | HPLC, NH₂-column | 1.3 | 0.1 | 0.1 |

The melting point (m.p.) was measured by using European Pharmacopoeia process same way as in the EP 1664352. The melting point of the crystals was 139.6-140.8° C.

In this specification, the following definitions have been used:

Fucose refers to monomeric fucose, which is typically L-fucose.

HMO refers to human milk oligosaccharide.

2′-FL refers to 2′-fucosyllactose.

3-FL refers to 3-fucosyllactose.

DFL, DiFL and LDFT refer to difucosyllactose.

HMO hydrolysate refers to a hydrolysed HMO, such as 2′-FL, 3-FL or LDFT or mixtures thereof.

Galactose and glucose refer to D-galactose and D-glucose.

IU refers to International Unit, which is the amount of enzyme consuming 1 μmol substrate per minute under standard conditions

SAC refers to a strong acid cation exchange resin.

WAC refers to a weak acid cation exchange resin.

DVB refers to divinylbenzene.

ACN refers to acetonitrile.

DS refers to a dry substance content expressed as % by weight.

RDS refers to DS content according to the correlation between refractometric index and DS.

SMB refers to chromatographic simulated moving bed process.

IX or IEX refer to ion exchange process

BV/h refers to the volume flow rate through an ion exchange material contained in a column or operating unit. BV refers to bed volume which is volume of ion exchange material of specified ionic form contained in a column or operating unit.

“A fraction enriched in L-fucose” or “an L-fucose fraction” refers to a fraction recovered from a chromatographic separation system or membrane filtration and having a greater content of fucose on DS than the solution used as the feed.

RDS refers to a refractometric dry substance content, expressed as percent by weight.

Purity refers to the content of a component (such as L-fucose) on DS or RDS. The Area % calculation procedure reports the area of each peak in the chromatogram as a percentage of the total area of all peaks.

Theoretical yield refers to the maximum amount of L-fucose that could be formed from the given amounts of hydrolyzed HMO.

The words “comprise”, “comprises” and “comprising” are to be interpreted inclusively rather than exclusively. This interpretation is intended to be the same as the interpretation that these words are given under United States patent law at the time of this filing.

The singular forms “a” and “an” are intended to include plural referents unless the context dictates otherwise. Thus, for example, a reference to the presence of “a microorganism” does not exclude the presence of multiple microorganisms unless the context dictates otherwise.

Any reference cited in this specification is incorporated by reference into this specification.

1. A process for making L-fucose, wherein the process comprises:
hydrolyzing a human milk oligosaccharide, which contains one or more L-fucose moieties in its structure, to form a hydrolysate comprising L-fucose, lactose, galactose and glucose;
subjecting the hydrolysate to one or more purification steps comprising chromatographic separation and/or nanofiltration;
recovering a fraction enriched in L-fucose from the purification; and
subjecting the fucose-enriched fraction to spray-drying and/or crystallization to form a purified L-fucose solid.

2. The process according to claim 1, wherein the human milk oligosaccharide is selected from 2′-fucosyllactose (2′-FL), 3-fucosyllactose (3-FL) and difucosyllactose (LDFT).

3. The process according to claim 1, wherein the hydrolysis comprises contacting the human milk oligosaccharide with an acid.

4. The process according to claim 1, wherein the hydrolysis comprises contacting the human milk oligosaccharide with an ion exchange column comprising a strong acid cation (SAC) ion exchange resin in H⁺-ion form.

5. The process according to claim 1, wherein the hydrolysis comprises hydrolyzing the human milk oligosaccharide at a pH of from 1.00 to 2.00.

6. The process according to claim 1, wherein the hydrolysis comprises hydrolyzing the human milk oligosaccharide at a temperature of from 70 to 140° C.

7. The process according to claim 1, wherein the hydrolysis is carried out with a residence time of from 1 to 24 hr.

8. The process according to claim 1, wherein the hydrolysis comprises contacting the human milk oligosaccharide with an enzyme.

9. The process according to claim 1, wherein the hydrolysis produces an aqueous hydrolysate with an L-fucose yield equaling more than 50% based on the human milk oligosaccharide that is hydrolyzed.

10. The process according to claim 1, wherein the hydrolysis produces an aqueous hydrolysate with galactose and glucose yields equaling less than 20% based on potential lactose that could be obtained during the hydrolysis.

11. The process according to claim 1, wherein the hydrolysis produces an aqueous hydrolysate having an L-fucose content of greater than 10% on DS.

12. The process according to claim 1, wherein:
the purification comprises chromatographic separation, and
the chromatographic separation comprises use of a resin selected from a strong acid cation resin (SAC) and a weak acid cation resin (WAC).

13. The process according to claim 1, wherein:
the purification comprises chromatographic separation, and
the chromatographic separation forms a chromatographic separation product comprising an L-fucose content that is greater than 60% on DS.

14. The process according to claim 1, wherein:
the purification comprises chromatographic separation, and
the chromatographic separation forms a chromatographic separation product having an L-fucose yield of greater than 70% based on the L-fucose in the hydrolysate.

15. The process according to claim 1, wherein:
the purification comprises nanofiltration, and
the nanofiltration forms a nanofiltration product having an L-fucose yield of greater than 50% based on the L-fucose in the hydrolysate.

16. The process according to claim 1, wherein the fucose-enriched fraction is subjected to crystallization to form a crystalline L-fucose product.

17. The process according to claim 1, wherein:
the fucose-enriched fraction is subjected to crystallization; and
the crystallization comprises crystallization in a solvent selected from water, an alcohol, and a mixture of water and an alcohol.

18. The process according to claim 1, wherein:
the fucose-enriched fraction is subjected to crystallization; and
the crystallization forms a crystalline L-fucose product having a purity of greater than 90% on DS.

19. An L-fucose product obtained from a process of claim 1.

20. The L-fucose product of claim 19, wherein the product comprises crystalline L-fucose.

21-22. (canceled)
